(19) United States
(12) Patent Application Publication
(10) Pub. No.: US 2024/0390892 A1
(43) Pub. Date: Nov. 28, 2024

(54) MICROFLUIDIC SUBSTRATE AND MICROFLUIDIC CHIP

(71) Applicants: Beijing BOE Technology Development Co., Ltd., Beijing (CN); BOE TECHNOLOGY GROUP CO., LTD., Beijing (CN)

(72) Inventors: Zhukai Liu, Beijing (CN); Ruijun Deng, Beijing (CN); Ding Ding, Beijing (CN)

(21) Appl. No.: 18/043,421

(22) PCT Filed: Oct. 28, 2021
(86) PCT No.: PCT/CN2021/127002

§ 371 Date: Feb. 28, 2023

(51) Int. Cl. B01L 3/00; B01L 7/00

(52) U.S. Cl. CPC B01L 3/502715; B01L 7/52; B01L 2200/16; B01L 2300/0654; B01L 2300/0893; B01L 2300/165; B01L 2300/18

(57) ABSTRACT

The present disclosure provides a microfluidic substrate and a microfluidic chip including the microfluidic substrate. The microfluidic substrate includes a plurality of microcavities arranged in an array, at least some of the plurality of microcavities are through holes, and a tangent plane at each of at least some points on a sidewall of each microcavity forms a non-perpendicular angle with a reference plane where the microfluidic substrate is located.

### TECHNICAL FIELD

The present disclosure relates to the field of biomedical detection, and in particular, to a microfluidic substrate and a microfluidic chip including the microfluidic substrate.

### BACKGROUND

Polymerase Chain Reaction (PCR) is a molecular biology technique used to amplify specific DNA fragments, which can replicate a small amount of deoxyribonucleic acid (DNA) in large quantities and hence greatly increase the number of DNA. Digital polymerase chain reaction (digital PCR, dPCR) technology is a quantitative analysis technology developed on the basis of PCR that can provide digital DNA quantitative information, and greatly improves the sensitivity and precision combined with the microfluidic technology. In the dPCR technique, nucleic acid samples are sufficiently diluted so that the number of the target molecule (i.e., the DNA template) in each reaction unit is less than or equal to one. PCR amplification is performed on the target molecule in each reaction unit, and after the amplification, the fluorescence signal of each reaction unit is statistically analyzed, so as to realize the absolute quantitative detection of single-molecule DNA. Due to the advantages of high sensitivity, strong specificity, high detection throughput, accurate quantification, and so on, dPCR has been widely used in clinical diagnosis, gene instability analysis, single-cell gene expression, environmental microbial detection, and prenatal diagnosis.

### SUMMARY

According to an aspect of the present disclosure, a microfluidic substrate is provided. The microfluidic substrate comprises a plurality of microcavities arranged in an array, at least some of the plurality of microcavities are through holes, and a tangent plane at each of at least some points on a sidewall of each microcavity forms a non-perpendicular angle with a reference plane where the microfluidic substrate is located.

In some embodiments, the sidewall of each microcavity comprises at least one of a curved surface and an inclined surface, the inclined surface is non-perpendicular to the reference plane.

In some embodiments, each of the plurality of microcavities is a through hole, and each microcavity comprises a top opening and a bottom opening.

In some embodiments, a shape of each microcavity is a circular truncated cone or a regular prismoid, and an area of an orthographic projection of the top opening of each microcavity on the reference plane is larger than an area of an orthographic projection of the bottom opening of each microcavity on the reference plane.

In some embodiments, an angle between a normal of any point on the sidewall of each microcavity and a reference line is 82°˜85°, the reference line is perpendicular to the reference plane.

In some embodiments, the microfluidic substrate further comprises a hydrophobic layer. The hydrophobic layer is on a first surface and a second surface of the microfluidic substrate which are opposite, a portion of the hydrophobic layer on the first surface comprises a plurality of first vias, and a portion of the hydrophobic layer on the second surface comprises a plurality of second vias. The plurality of first vias and the plurality of second vias correspond to the plurality of microcavities one by one respectively, the orthographic projection of the top opening of each of the plurality of microcavities on the reference plane is within an orthographic projection of a first via corresponding to the microcavity on the reference plane, and the orthographic projection of the bottom opening of each of the plurality of microcavities on the reference plane overlaps an orthographic projection of the second via corresponding to the microcavity on the reference plane.

In some embodiments, a shape of each microcavity is axisymmetric about a symmetry axis, the symmetry axis is parallel to the reference plane.

In some embodiments, each microcavity comprises a first portion and a second portion stacked on and penetrating through each other, the first portion and the second portion are axisymmetric about the symmetry axis, and a shape of each of the first portion and the second portion is one of a circular truncated cone and a regular prismoid, and an area of an orthographic projection of a first top opening of the first portion on the reference plane is larger than an area of an orthographic projection of a second bottom opening of the first portion on the reference plane, an area of an orthographic projection of a third top opening of the second portion on the reference plane is smaller than an area of an orthographic projection of a fourth bottom opening of the second portion on the reference plane.

In some embodiments, each microcavity further comprises a third portion between the first portion and the second portion and connecting the first portion and the second portion, the second bottom opening of the first portion is a fifth top opening of the third portion, the third top opening of the second portion is a sixth bottom opening of the third portion, and the third portion is axisymmetric about the symmetry axis.

In some embodiments, the shape of each of the first portion and the second portion is the circular truncated cone, and a shape of the third portion is a cylinder, or the shape of each of the first portion and the second portion is a regular quadrangular prismoid, and a shape of the third portion is a cuboid.

In some embodiments, the shape of each of the first portion and the second portion is the circular truncated cone, and a shape of the third portion is a curved surface body, a vertical distance from any point on a sidewall of the third portion to a reference line is greater than a radius of the fifth top opening of the third portion, the reference line passes through centers of the fifth top opening and the sixth bottom opening of the third portion and is perpendicular to the reference plane.

In some embodiments, a shape of the top opening of each microcavity is a circle, and a diameter of the circle is 110˜130 μm.

In some embodiments, each microcavity comprises a fourth portion and a fifth portion stacked on and penetrating through each other, the fourth portion and the fifth portion are axisymmetric about the symmetry axis, and a shape of each of the fourth portion and the fifth portion is a curved surface body, a shape of each of the top opening and the bottom opening of each microcavity is a circle, a vertical distance from any point on the sidewall of each microcavity to a reference line is greater than a radius of the top opening, and the reference line passes through centers of the top opening and the bottom opening and is perpendicular to the reference plane.

In some embodiments, a diameter of the top opening is 210˜230 μm.

In some embodiments, a depth of each microcavity is 300 μm.

In some embodiments, others of the plurality of microcavities are blind holes.

In some embodiments, a shape of the blind hole is a curved surface body, the blind hole comprises an opening, a sidewall and a bottom, the opening of the blind hole is a top opening of the microcavity and a shape of the opening is a circle, a vertical distance from any point on the sidewall of the blind hole to a reference line is greater than a radius of the top opening, the reference line passes through the center of the top opening and is perpendicular to the reference plane.

In some embodiments, a depth of the blind hole is 50˜100 μm, and a diameter of the opening of the blind hole is 110˜130 μm.

In some embodiments, a ratio of the maximum value of the vertical distance to the radius of the top opening is 1.2:1.

In some embodiments, a distance between two adjacent microcavities in the plurality of microcavities is 20˜50 μm.

In some embodiments, the plurality of microcavities are arranged in a glass substrate of the microfluidic substrate.

In some embodiments, the microfluidic substrate further comprises a heating electrode, the heating electrode is arranged in a region between two adjacent microcavities on at least one of the first surface and the second surface of the microfluidic substrate which are opposite.

In some embodiments, the microfluidic substrate further comprises a hydrophobic layer, the heating electrode is arranged in the region between two adjacent microcavities on both the first surface and the second surface of the microfluidic substrate which are opposite, and the hydrophobic layer is on a side of the heating electrode away from the first surface and a side of the heating electrode away from the second surface.

In some embodiments, the microfluidic substrate further comprises: a first dielectric layer on a side of the heating electrode close to the first surface and a side of the heating electrode close to the second surface; a second dielectric layer on a side of the first dielectric layer away from the first surface and a side of the first dielectric layer away from the second surface; and a conductive layer between the first dielectric layer and the second dielectric layer and arranged on the periphery of the microfluidic substrate, the conductive layer being electrically connected to the heating electrode through vias in the second dielectric layer.

According to another aspect of the present disclosure, a microfluidic chip comprising the microfluidic substrate described in any one of the preceding embodiments is provided.

In some embodiments, the microfluidic chip further comprises a counter substrate assembled with the microfluidic substrate and an encapsulant between the microfluidic substrate and the counter substrate.

### BRIEF DESCRIPTION OF THE DRAWINGS

In order to describe the technical solutions in the embodiments of the present disclosure more clearly, the accompanying drawings required in the embodiments will be briefly introduced below. Obviously, the drawings in the following description are only some embodiments of the present disclosure. For those of ordinary skill in the art, other drawings can also be obtained from these drawings without any creative effort.

FIG. 1 illustrates a plurality of microcavities of a microfluidic substrate according to an embodiment of the present disclosure;

FIG. 2A illustrates a cross-sectional view of a partial structure of a microfluidic substrate according to an embodiment of the present disclosure;

FIG. 2B illustrates a schematic structural diagram of the microcavity in FIG. 2A;

FIG. 2C illustrates another schematic structural diagram of the microcavity in FIG. 2A;

FIG. 3A illustrates a cross-sectional view of a partial structure of a microfluidic substrate according to an embodiment of the present disclosure;

FIG. 3B illustrates a schematic structural diagram of the microcavity in FIG. 3A;

FIG. 3C illustrates another schematic structural diagram of the microcavity in FIG. 3A;

FIG. 4A illustrates a cross-sectional view of a partial structure of a microfluidic substrate according to an embodiment of the present disclosure;

FIG. 4B illustrates a schematic structural diagram of the microcavity in FIG. 4A;

FIG. 4C illustrates another schematic structural diagram of the microcavity in FIG. 4A;

FIG. 5A illustrates a cross-sectional view of a partial structure of a microfluidic substrate according to an embodiment of the present disclosure;

FIG. 5B illustrates a schematic structural diagram of the microcavity in FIG. 5A;

FIG. 6A illustrates a cross-sectional view of a partial structure of a microfluidic substrate according to an embodiment of the present disclosure;

FIG. 6B illustrates a schematic structural diagram of the microcavity in FIG. 6A;

FIG. 7A illustrates through holes and blind holes of a plurality of microcavities of a microfluidic substrate according to an embodiment of the present disclosure;

FIG. 7B illustrates a schematic cross-sectional view taken along line II′ in FIG. 7A;

FIG. 8A illustrates an arrangement of a plurality of microcavities of a microfluidic substrate according to an embodiment of the present disclosure;

FIG. 8B illustrates another arrangement of a plurality of microcavities of a microfluidic substrate according to an embodiment of the present disclosure;

FIG. 9 illustrates a cross-sectional view of a partial structure of a microfluidic substrate according to an embodiment of the present disclosure;

FIG. 10 illustrates a schematic structural diagram of the hydrophobic layer in FIG. 9; and

FIG. 11 illustrates a schematic structural diagram of a microfluidic chip according to an embodiment of the present disclosure.

### DETAILED DESCRIPTION OF THE DISCLOSURE

The technical solutions in the embodiments of the present disclosure will be clearly and completely described below with reference to the accompanying drawings in the embodiments of the present disclosure. Obviously, the described embodiments are only some, but not all, embodiments of the present disclosure. Based on the embodiments in the present disclosure, all other embodiments obtained by those of ordinary skill in the art without creative efforts shall fall within the protection scope of the present disclosure.

dPCR has been widely used in clinical diagnosis, single-cell analysis, early cancer diagnosis, gene instability analysis, environmental microbial detection and prenatal diagnosis due to its advantages of high sensitivity, strong specificity, high detection throughput, and accurate quantification. dPCR technology is an absolute quantification technology of nucleic acid molecules. Its principle can be roughly described as follows: the sample solution comprising the target nucleic acid molecules is fully diluted, and then the diluted sample solution is distributed into a large number of microcavities of the microfluidic chip, so that each microcavity comprises only one or zero nucleic acid molecule. PCR amplification of single molecule is performed in each microcavity to form a solution to be tested. Then a fluorescence microscope or flow cytometer is used to detect the fluorescence intensity of the solution to be tested in each microcavity, and finally the number (or concentration) of target nucleic acid molecules in the original sample can be calculated through the number of positive microcavities and Poisson distribution statistics, so as to achieve absolute quantification.

Microfluidic chip comprises multiple small-sized microcavities. At present, there are still many challenges in the application of the microfluidic chip based on microcavity structures. For example, the abundance of circulating tumor DNA (ctDNA) in blood is usually very low, and it is usually necessary to enrich the ctDNA when using the microfluidic chip for ctDNA detection. To reduce this operation, one can choose to increase the total reaction volume of the multiple microcavities of the microfluidic chip to increase the minimum detection limit of the microfluidic chip. Increasing the total reaction volume of the microcavity can improve the minimum detection limit of the microfluidic chip because, according to the Poisson distribution, it can generally be considered that c=−ln(b/n)/v, where c is the concentration of the sample detection target (unit: copies per microliter), b is the number of negative microcavities, n is the total number of microcavities, and v is the volume of a single microcavity (unit: microliter). It can be seen that the larger v is, the lower the minimum limit of detectable target concentration is. However, excessively increasing the volume of a single microcavity will affect the stability of the immobilization effect of the sample solution in the microcavity. Due to the excessively large volume of the microcavity, on the one hand, the ratio of the diameter of the opening of the microcavity to the depth of the microcavity may be too large, so that the encapsulated oil can easily flush the sample solution in the microcavity to the outside of the microcavity or into another adjacent microcavity, resulting in waste or crosstalk of the sample solution; on the other hand, the microcavity with a large volume can accommodate more doses of sample solution, but too much sample solution easily flows out from the bottom opening of the microcavity under the influence of its own gravity, resulting in an inability to keep stably in the microcavity. In addition, in the related art, the sidewall of the microcavity is usually a vertical wall, that is, the sidewall of the microcavity is perpendicular to the surface of the microfluidic chip. Such a steep sidewall is unfavorable for the sample solution to enter the microcavity, which causes the sample solution to enter the microcavity very slowly or even stagnate on the surface of the microfluidic chip, so that the injection efficiency is reduced and even the trace amount of sample solution is wasted.

In order to solve the problems existing in the related art, the embodiments of the present disclosure provide a microfluidic substrate. The microfluidic substrate provided by the embodiments of the present disclosure can not only perform dPCR detection and analysis on nucleic acids extracted from tumor tissue cells, peripheral blood samples, etc., but also be applied to digital analysis biological detection such as digital isothermal amplification, single-molecule immunity, which provides new options for popular medical fields such as single-cell analysis, early cancer diagnosis and prenatal diagnosis.

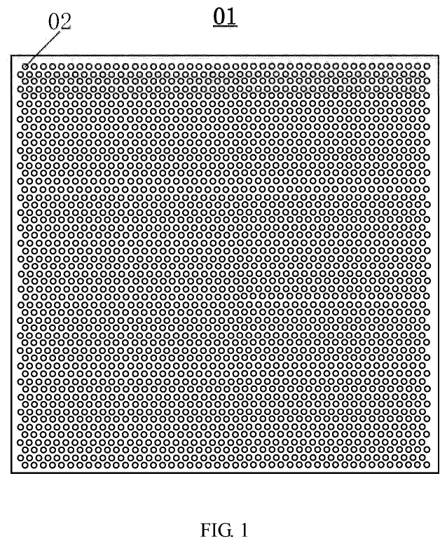

FIG. 1 illustrates a top plan view of a microfluidic substrate 01. As illustrated, the microfluidic substrate 01 comprises a plurality of microcavities 02 arranged in an array, at least some of the plurality of microcavities 02 are through holes, and a tangent plane at each of at least some points on a sidewall of each microcavity 02 forms a non-perpendicular angle with a reference plane where the microfluidic substrate 01 is located. All of the plurality of microcavities 02 may be through holes, or some of them may be through holes. The inner wall of the microcavity 02 usually has a hydrophilic effect due to its material (e.g. glass), while the microfluidic substrate 01 is usually provided with a hydrophobic layer with a hydrophobic effect on the surface. The sample solution in liquid form can be held within the microcavity 02 in the form of through hole due to the hydrophilicity, hydrophobicity and capillary action and due to the very small volume (on the order of microliters) of a single microcavity. It should be noted that, in the specification of the present application, the term “sidewall of the microcavity” refers to all the walls surrounding the periphery of the inner of the microcavity. The microcavity 02 comprises a top opening, a bottom opening and a sidewall, and the sidewall connects the top opening and the bottom opening. The sidewall, the top opening and the bottom opening of the microcavity 02 together constitute a reaction cavity of the microcavity 02 to accommodate the sample solution. The definition of “tangent plane” in mathematics textbooks is that under certain conditions, there are countless curves passing through a point M on the curved surface, and each curve has a tangent line at the point M. Under certain conditions, these tangent lines lie on the same plane, and this plane is called the tangent plane of the curved surface at the point M, and the point M is called the tangent point. Thus, the phrase “a tangent plane at each of at least some points on a sidewall of each microcavity 02 forms a non-perpendicular angle with a reference plane where the microfluidic substrate 01 is located” means that at least a portion of the sidewall of each microcavity 02 is not perpendicular to the reference plane (such as the horizontal plane) where the microfluidic substrate 01 is located. For example, all portions on the sidewall of the microcavity 02 may not be perpendicular to the reference plane, or one or more portions on the sidewall of the microcavity 02 are not perpendicular to the reference plane. In other words, at least a portion of the sidewall of each microcavity 02 has a certain inclination relative to the reference plane, and the inclination angle can be, for example, an acute angle (greater than 0° and less than 90°) or an obtuse angle (greater than 90° and less than 180°).

By designing the microcavity 02 as a through hole, under the capillary action, it is favorable for the sample solution to smoothly enter the inside of the microcavity 02, and will not stagnate on the surface of the microfluidic substrate 01, resulting in waste of the sample solution. In addition, the sample solution will inevitably generate some bubbles during the sampling process. The microcavity 02 is designed as the through hole, the gas can be discharged from the bottom opening of the microcavity 02, so as to prevent the bubbles from remaining in the microcavity 02, so as not to affect the subsequent fluorescence detection of the sample solution. By making at least a portion of the sidewall of the microcavity 02 not perpendicular to the reference plane where the microfluidic substrate 01 is located, the slope of the sidewall of the microcavity 02 relative to the reference plane can be reduced, which is beneficial for the sample solution to quickly enter the inside of the microcavity 02 along the sidewall without to stagnating on the surface of the microfluidic substrate 01, thereby improving the sample injection efficiency and the utilization of the sample solution.

In some embodiments, the sidewall of each microcavity 02 comprises at least one of a curved surface and a sloped surface, and the sloped surface is not perpendicular to the reference plane. The curved surface can be a curved surface with arbitrary curvature (e.g., varying curvature), such as an arc surface, a spherical surface, etc., such a curved surface is not perpendicular to the reference plane. The sloped plane may be an inclined plane with a certain inclined angle to the reference plane.

In the following, several different shapes of the microcavity 02 and the arrangement of other layers will be described through several embodiments. It should be noted that, the following description is only used as examples to illustrate several different shapes of the microcavity 02, but not all possible shapes of the microcavity 02 are exhausted. Based on the embodiments in the present disclosure, all other embodiments obtained by those of ordinary skill in the art without creative efforts shall fall within the protection scope of the present disclosure.

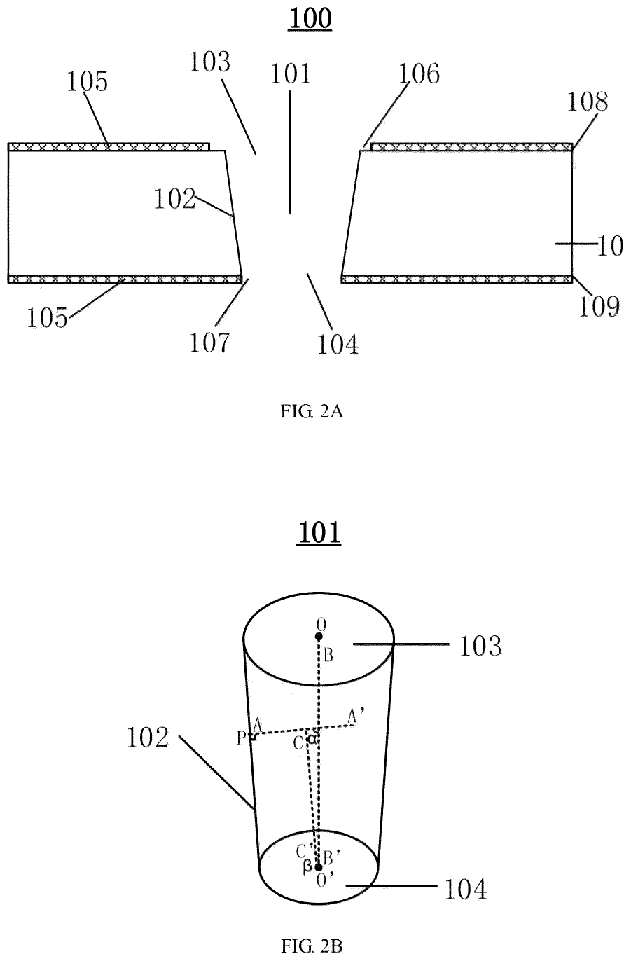

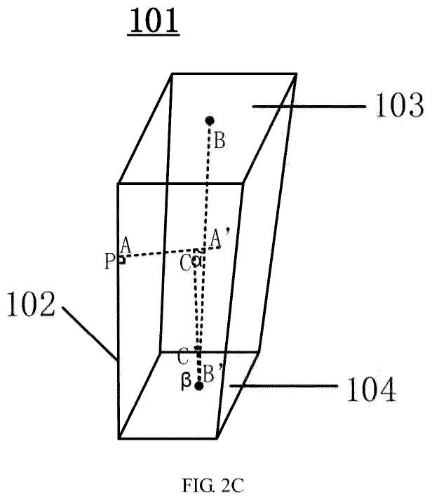

FIG. 2A illustrates a cross-sectional view of a portion of the microfluidic substrate 100, and the microfluidic substrate 100 comprises a plurality of microcavities 101. In this embodiment, each microcavity 101 of the microfluidic substrate 100 is a through hole. As illustrated, the microfluidic substrate 100 comprises a substrate 10, which may be any suitable material, comprising but not limited to glass, silicon, silicon oxide, and the like. In one example, the substrate 10 is a glass substrate in which the microcavities 101 are formed and through holes are formed by penetrating through the glass substrate. In other words, the glass substrate is etched to form a plurality of through holes, thereby forming a plurality of microcavities 101. The microcavity 101 comprises a top opening 103, a bottom opening 104, and a sidewall 102, and the sidewall 102 is inclined at a certain angle with respect to the reference plane where the microfluidic substrate 100 is located. By making the sidewall 102 have an inclination angle with respect to the reference plane, it is favorable for the sample solution to fully fill each microcavity 101 during the injection.

The shape of the microcavity 101 in FIG. 2A may be a circular truncated cone or a regular prismoid, and the regular prismoid may be a regular quadrangular prismoid, a regular pentagonal prismoid or any regular polygonal prismoid.

FIG. 2B illustrates, as an example, a microcavity 101 with a shape of circular truncated cone. The microcavity 101 comprises the top opening 103, the bottom opening 104 and the sidewall 102. Both the top opening 103 and the bottom opening 104 are circular, the top opening 103 has a center O, the bottom opening 104 has a center O′, and the area of the orthographic projection of the top opening 103 on the reference plane is larger than the area of the orthographic projection of the bottom opening 104 on the reference plane. The sidewall 102 has an inclination angle with respect to the reference plane. As illustrated in the figure, any point P on the sidewall 102 of the microcavity 101 has a normal line AN, which forms a right triangle with the first reference line BB′ and the second reference line CC′, wherein the first reference line BB′ is perpendicular to the reference plane (also perpendicular to the plane where the top opening 103 and the bottom opening 104 are located), and the second reference line CC′ is parallel to the generatrix of the circular truncated cone. The angle between the normal line AA and the first reference line BB′ is α, the angle between the first reference line BB′ and the second reference line CC′ is β, α and β are complementary angles to each other, α can be an acute angle with any appropriate numerical value, similarly, β can also be an acute angle with any appropriate numerical value, as long as the two are satisfied to add up to 90 degrees. In an example, the angle α between the normal line AA′ and the first reference line BB′ is 82°-85°, that is, the angle Q between the first reference line BB′ and the second reference line CC′ is 5°-8°. Therefore, it can be roughly considered that the slope angle of the sidewall of the microcavity 101 is 5°-8°. As mentioned above, by making the sidewall 102 have a certain inclination angle with respect to the reference plane, it is beneficial for the sample solution to flow into the inside of the microcavity 101 along the inclined sidewall 102 of the microcavity 101, so that the sample solution can fully fill each microcavity 101.

FIG. 2C illustrates, as another example, a microcavity 101 with a shape of a regular quadrangular prismoid. The microcavity 101 comprises the top opening 103, the bottom opening 104 and the sidewall 102. Both the top opening 103 and the bottom opening 104 are square, and the area of the orthographic projection of the top opening 103 on the reference plane is larger than the area of the orthographic projection of the bottom opening 104 on the reference plane. The sidewall 102 comprises four sides, which are congruent isosceles trapezoids. The sidewall 102 has an inclination angle with respect to the reference plane. As illustrated in the figure, any point P on the sidewall 102 of the microcavity 101 has a normal line AA′, which forms a right triangle with the first reference line BB′ and the second reference line CC′, wherein the first reference line BB′ is perpendicular to the reference plane (also perpendicular to the plane where the top opening 103 and the bottom opening 104 are located), and the second reference line CC′ is parallel to the side edges of the regular quadrangular prismoid. The angle between the normal line AA and the first reference line BB′ is α, the angle between the first reference line BB′ and the second reference line CC′ is β, α and β are complementary angles to each other, a can be an acute angle with any appropriate numerical value, similarly, β can also be an acute angle with any appropriate numerical value, as long as the two are satisfied to add up to 90 degrees. In an example, the angle α between the normal line AA′ and the first reference line BB′ is 82°-85°, that is, the angle β between the first reference line BB′ and the second reference line CC′ is 5°-8°. Therefore, it can be considered that the slope angle of each side surface of the sidewall 102 of the microcavity 101 is 5°-8°. As mentioned above, by making the sidewall 102 have a certain inclination angle with respect to the reference plane, it is beneficial for the sample solution to flow into the inside of the microcavity 101 along the inclined sidewall 102 of the microcavity 101, so that the sample solution can fully fill each microcavity 101.

The thickness of the substrate 10 is about 300 μm, so the depth of the microcavity 101 is about 300 μm. In some embodiments, the density of the microcavities 101 of the microfluidic substrate 100 is about 9000/cm². In the embodiment in which the top opening 103 of the microcavity 101 is circular, the diameter of the top opening 103 is 110-130 μm, such as 110 μm, 120 μm, 130 μm. The size of the microfluidic substrate 100 can be any appropriate size, and the number of the microcavities 101 can be any appropriate number. The embodiments of the present disclosure do not specifically limit the size of the microfluidic substrate 100 and the number of the microcavities 101. In an example, the size of the microfluidic substrate 100 is 5 cm*5 cm, and the number of the microcavities 101 is 100*100.

Referring back to FIG. 2A, the microfluidic substrate 100 may further comprise a hydrophobic layer 105. The microfluidic substrate 100 comprises the opposite first surface 108 and second surface 109. The hydrophobic layer 105 is on the opposite first surface 108 and the second surface 109 of the microfluidic substrate 100. The hydrophobic layer 105 has hydrophobic and oleophilic properties, and the material of the hydrophobic layer 105 can be resin or silicon nitride. Since the sample solution is an aqueous liquid, disposing the hydrophobic layer 105 on the first surface 108 and the second surface 109 of the microfluidic substrate 100 can prevent the aqueous sample solution from staying on the surface of the microfluidic substrate 100 and facilitate its entry into the inside of the microfluidic substrate 100. The portion of the hydrophobic layer 105 located on the first surface 108 comprises a plurality of first vias 106, and the portion of the hydrophobic layer 105 located on the second surface 109 comprises a plurality of second vias 107. The plurality of first vias 106 and the plurality of second vias 107 are in one-to-one correspondence with the plurality of microcavities 101, that is, the number of the first vias 106 is the same as the number of the microcavities 101, and the number of the second vias 107 is also the same as the number of the microcavities 101. The shape of the first via 106 and the second via 107 is adapted to the shape of the top opening 103 and the bottom opening 104 of the microcavity 101. For example, when the shapes of the top opening 103 and the bottom opening 104 of the microcavity 101 are circular, respectively, the shapes of the first via 106 and the second via 107 are correspondingly circular; when the shapes of the top opening 103 and the bottom opening 104 of the microcavity 101 are respectively regular polygons, the shapes of the first via 106 and the second via 107 are correspondingly regular polygons. The orthographic projection of the top opening 103 of each microcavity 101 on the reference plane is located within the orthographic projection of a first via 106 corresponding to the microcavity 101 on the reference plane, and the orthographic projection of the bottom opening 104 of each microcavity 101 on the reference plane overlaps with the orthographic projection of a second via 107 corresponding to the microcavity 101 on the reference plane, for example, the two orthographic projections may completely overlap. In other words, the edge of the first via 106 of the hydrophobic layer 105 is separated from the edge of the top opening 103 of the microcavity 101 by a certain distance in the horizontal direction, the edge of the second via 107 of the hydrophobic layer 105 is substantially coincident with the edge of the bottom opening 104 of the microcavity 101 in the vertical direction. By designing the first via 106 and the second via 107 of the hydrophobic layer 105 in this way, the sample solution can be facilitated to enter the inside of the microcavity 101 along the sidewall 102 of the microcavity 101 and be difficult to flow out of the microcavity 101.

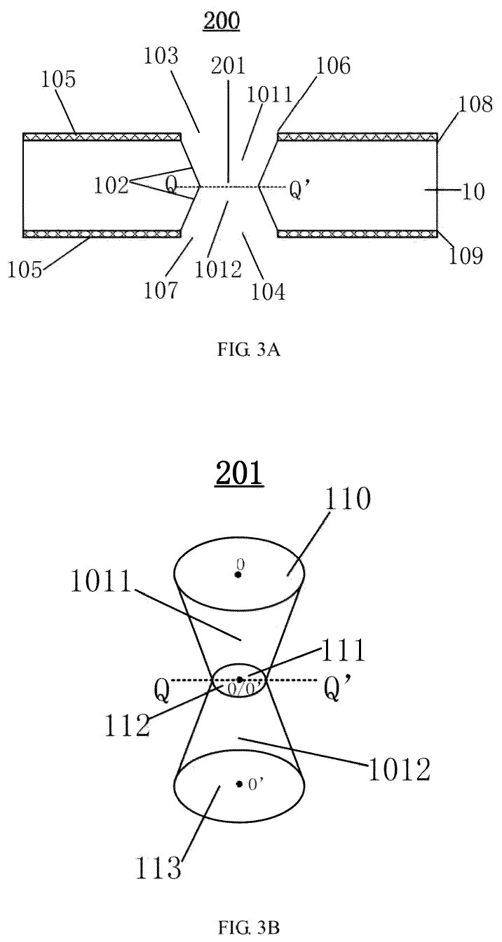

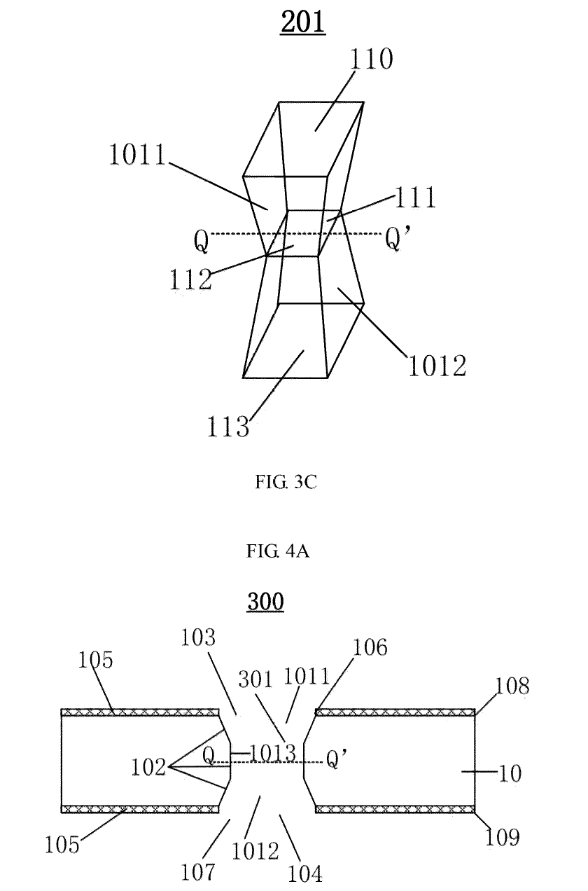

FIG. 3A illustrates a cross-sectional view of a portion of the microfluidic substrate 200, and the microfluidic substrate 200 comprises a plurality of microcavities 201. In this embodiment, each microcavity 201 of the microfluidic substrate 200 is a through hole. The difference from the microfluidic substrate 100 in FIG. 2A is that the shape of the microcavity 201 of the microfluidic substrate 200 in FIG. 3A is different from the shape of the microcavity 101 of the microfluidic substrate 100 in FIG. 2A, and the arrangement of the hydrophobic layer 105 of the microfluidic substrate 200 in FIG. 3A is slightly different from the arrangement of the hydrophobic layer 105 of the microfluidic substrate 100 in FIG. 2A.

As illustrated in FIG. 3A, each microcavity 201 comprises a first portion 1011 and a second portion 1012 stacked on each other and penetrating through each other, the first portion 1011 and the second portion 1012 being axisymmetric about the symmetry axis QQ′, which is parallel to the reference plane. That is, the first portion 1011 completely overlaps with the second portion 1012 after being flipped 180 degrees with respect to the symmetry axis QQ′. The shape of the first portion 1011 and the second portion 1012 may be one of a circular truncated cone and a regular prismoid, and the regular prismoid may be a regular quadrangular prismoid, a regular pentagonal prismoid or any regular polygonal prismoid.

FIG. 3B illustrates the shape of the microcavity 201 as an example, wherein the first portion 1011 and the second portion 1012 of the microcavity 201 are both circular truncated cone. The first portion 1011 comprises a first top opening 110 and a second bottom opening 111, the second portion 1012 comprises a third top opening 112 and a fourth bottom opening 113, the second bottom opening 111 of the first portion 1011 and the third top opening 112 of the second portion 1012 are the same opening, that is, the two completely overlap. The first top opening 110 and the second bottom opening 111 of the first portion 1011 and the third top opening 112 and the fourth bottom opening 113 of the second portion 1012 are all circular. The first top opening 110 of the first portion 1011 is the top opening 103 of the microcavity 201, and the fourth bottom opening 113 of the second portion 1012 is the bottom opening 104 of the microcavity 201.

FIG. 3C illustrates the shape of the microcavity 201 as another example, wherein both the first portion 1011 and the second portion 1012 of the microcavity 201 are in the shape of a regular quadrangular prismoid. The first portion 1011 comprises a first top opening 110 and a second bottom opening 111, the second portion 1012 comprises a third top opening 112 and a fourth bottom opening 113, the second bottom opening 11I of the first portion 1011 and the third top opening 112 of the second portion 1012 are the same opening, that is, the two completely overlap. The first top opening 110 and the second bottom opening 111 of the first portion 1011 and the third top opening 112 and the fourth bottom opening 113 of the second portion 1012 are all square. The sidewall 102 of the microcavity 201 comprises eight side surfaces, and the eight side surfaces are congruent isosceles trapezoids. The first top opening 110 of the first portion 1011 is the top opening 103 of the microcavity 201, and the fourth bottom opening 113 of the second portion 1012 is the bottom opening 104 of the microcavity 201.

In FIG. 3B or FIG. 3C, the area of the orthographic projection of the first top opening 110 of the first portion 1011 of the microcavity 201 on the reference plane is larger than the area of the orthographic projection of the second bottom opening 111 on the reference plane, the area of the orthographic projection of the third top opening 112 of the second portion 1012 on the reference plane is smaller than the area of the orthographic projection of the fourth bottom opening 113 on the reference plane. The area of the first top opening 110 is equal to the area of the fourth bottom opening 113, and the area of the second bottom opening 111 is equal to the area of the third top opening 112, so that the first portion 1011 and the second portion 1012 are axisymmetric about the symmetry axis QQ′.

The thickness of the substrate 10 is about 300 μm, so the depth of the microcavity 201 is about 300 μm. In some embodiments, the density of the microcavities 201 of the microfluidic substrate 200 is about 9000/cm². In the embodiment in which the top opening 103 of the microcavity 201 is circular, the diameter of the top opening 103 is 110-130 μm, such as 110 μm, 120 μm, 130 μm. The size of the microfluidic substrate 200 can be any appropriate size, and the number of the microcavities 201 can be any appropriate number. The embodiments of the present disclosure do not specifically limit the size of the microfluidic substrate 200 and the number of the microcavities 201. In an example, the size of the microfluidic substrate 200 is 5 cm*5 cm, and the number of the microcavities 201 is 100*100.

Similar to the microcavity 101, the sidewall 102 of the microcavity 201 has a slope angle with respect to the reference plane. In an example, the slope angle of the sidewall 102 of the microcavity 201 is 5°-8°. By making the sidewall 102 have an inclination angle with respect to the reference plane, it is beneficial for the sample solution to flow into the microcavity 201 along the inclined sidewall 102 of the microcavity 201, so that the sample solution can fully fill each microcavity 201.

Regarding the hydrophobic layer 105, the arrangement of the hydrophobic layer 105 of the microfluidic substrate 200 illustrated in FIG. 3A is basically the same as the arrangement of the hydrophobic layer 105 of the microfluidic substrate 100 illustrated in FIG. 2A. The only difference is that the orthographic projection of the top opening 103 of each microcavity 201 on the reference plane overlaps with the orthographic projection of a first via 106 corresponding to the microcavity 201 on the reference plane, and the orthographic projection of the bottom opening 104 of each microcavity 201 on the reference plane overlaps with the orthographic projection of a second via 107 corresponding to the microcavity 201 on the reference plane.

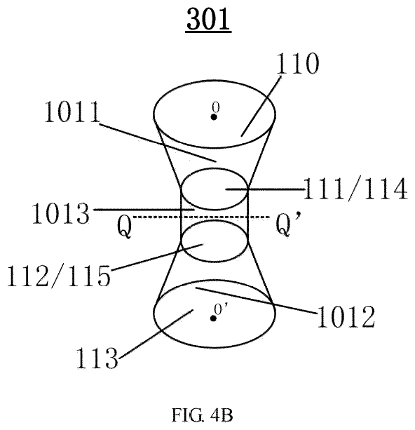

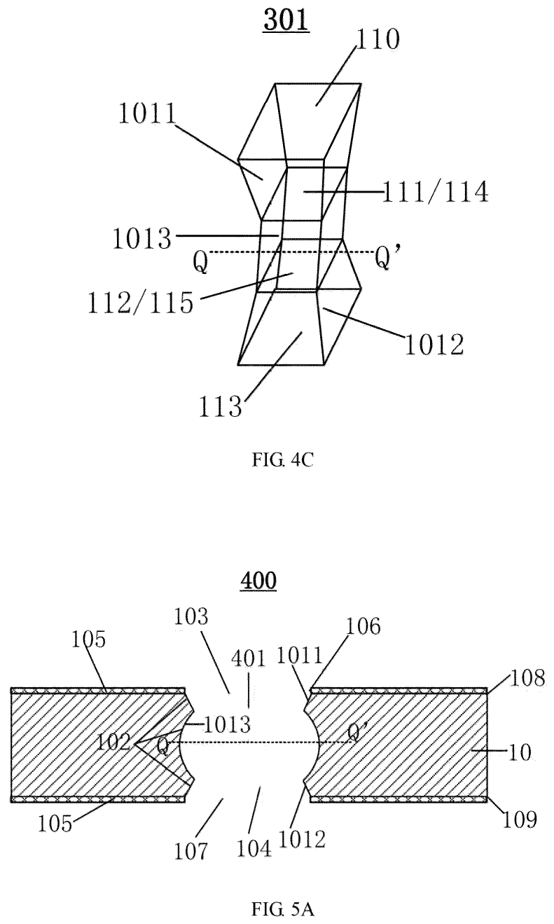

FIG. 4A illustrates a cross-sectional view of a portion of a microfluidic substrate 300, and the microfluidic substrate 300 comprises a plurality of microcavities 301. FIG. 4B and FIG. 4C illustrate two shapes of the microcavity 301 as examples. The microfluidic substrate 300 illustrated in FIGS. 4A-4C has substantially the same construction as the microfluidic substrate 200 illustrated in FIGS. 3A-3C, and therefore the same reference numerals are used to refer to the same components. Therefore, the detailed functions of the components in FIGS. 4A-4C with the same reference numerals as those in FIGS. 3A-3C can refer to the description of FIGS. 3A-3C, which will not be repeated here, and only the differences will be described below.

As illustrated in FIGS. 4A-4C, each microcavity 301 further comprises a third portion 1013 between the first portion 1011 and the second portion 1012 and connecting the first portion 1011 and the second portion 1012. The third portion 1013 is axisymmetric about the symmetry axis QQ′. For the first portion 1011 and the second portion 1012 in FIGS. 4A-4C, the reference may be made to the description of the first portion 1011 and the second portion 1012 in FIGS. 3A-3C. Since the first portion 1011 and the second portion 1012 are also axisymmetric about the symmetry axis QQ′, the microcavity 301 formed by the first portion 1011, the second portion 1012 and the third portion 1013 is axisymmetric about the symmetry axis QQ′.

As illustrated in FIG. 4B, when the shapes of the first portion 1011 and the second portion 1012 are circular truncated cones, the shape of the third portion 1013 may be a cylinder whose sidewall is perpendicular to the reference plane. As illustrated in FIG. 4C, when the shapes of the first portion 1011 and the second portion 1012 are regular quadrangular prismoids, the shape of the third portion 1013 may be a cuboid whose sidewall is perpendicular to the reference plane. The third portion 1013 comprises a fifth top opening 114 and a sixth bottom opening 115, and in FIG. 4C, the fifth top opening 114 and the sixth bottom opening 115 are both square. The cuboid comprises a rectangular box or a cube, when the height (i.e. the side edge of the third portion 1013 between the first portion 1011 and the second portion 1012) of the cuboid is equal to the side lengths of the fifth top opening 114 and the sixth bottom opening 115, the cuboid is a cube; when the height of the cuboid is not equal to the side lengths of the fifth top opening 114 and the sixth bottom opening 115, the cuboid is the rectangular box.

As illustrated in FIG. 4B and FIG. 4C, the second bottom opening 111 of the first portion 1011 of the microcavity 301 is the fifth top opening 114 of the third portion 1013, and the third top opening 112 of the second portion 1012 of the microcavity 301 is the sixth bottom opening 115 of the third portion 1013.

Similar to the microcavity 201 in FIG. 3A, the sidewall 102 of the microcavity 301 in FIG. 4A has a slope angle with respect to the reference plane. In an example, the slope angle of the sidewall 102 of the microcavity 301 is 5°-8°. By making the sidewall 102 have an inclination angle with respect to the reference plane, it is favorable for the sample solution to flow into the microcavity 301 along the inclined sidewall 102 of the microcavity 301, so that the sample solution can fully fill each microcavity 301. In addition, compared with the microcavity 201 in FIG. 3A, the microcavity 301 in FIG. 4A has the middle portion 1013. The existence of the middle portion 1013 can increase the volume of the cavity on the one hand, so that each microcavity 301 can accommodate much sample solution, and more doses of reagents to be tested can be obtained after PCR amplification reaction, thereby improving the minimum detection limit of the microfluidic substrate 300. On the other hand, the sidewall of the third portion 1013 is perpendicular to the reference plane, so that the sample solution can not only smoothly enter the inside of the microcavity 301 along the inclined sidewall of the first portion 1011 of the microcavity 301 and fully fill each microcavity 301, but also be kept stably in the microcavity 301 due to the existence of the vertical sidewall of the third portion 1013, and is not easy to flow out of the microcavity 301.

The thickness of the substrate 10 is about 300 μm, that is, the depth of the microcavity 301 is about 300 μm. In some embodiments, the density of the microcavities 301 of the microfluidic substrate 300 is about 9000/cm². In the embodiment in which the top opening 103 of the microcavity 301 is circular, the diameter of the top opening 103 is 110-130 μm, such as 110 μm, 120 μm, 130 μm. The size of the microfluidic substrate 300 can be any appropriate size, and the number of the microcavities 301 can be any appropriate number. The embodiments of the present disclosure do not specifically limit the size of the microfluidic substrate 300 and the number of the microcavities 301. In an example, the size of the microfluidic substrate 300 is 5 cm*5 cm, and the number of the microcavities 301 is 100*100.

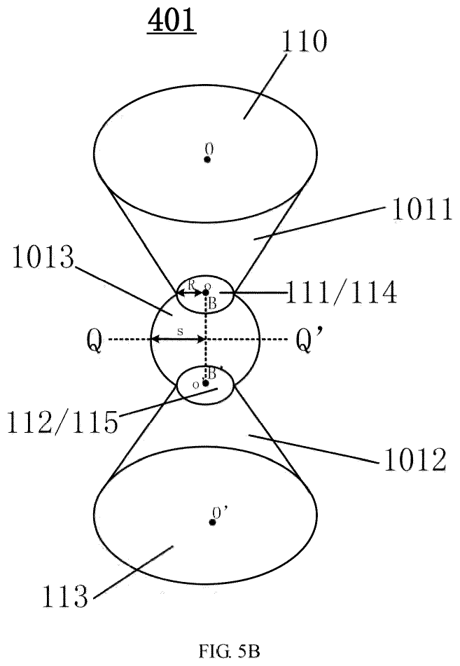

FIG. 5A illustrates a cross-sectional view of a portion of a microfluidic substrate 400, the microfluidic substrate 400 comprises a plurality of microcavities 401. FIG. 5B illustrates a shape of the microcavity 401 as an example. The microfluidic substrate 400 illustrated in FIGS. 5A-5B has substantially the same construction as the microfluidic substrate 200 illustrated in FIGS. 3A-3C, and therefore the same reference numerals are used to refer to the same components. Therefore, the detailed functions of the components in FIGS. 5A-5B with the same reference numerals as those in FIGS. 3A-3C can refer to the description of FIGS. 3A-3C, which will not be repeated here, and only the differences will be described below.

As illustrated in FIG. 5A, each microcavity 401 further comprises a third portion 1013 located between the first portion 1011 and the second portion 1012 and connecting the first portion 1011 and the second portion 1012, the third portion 1013 is axisymmetric about the symmetry axis QQ′. The first portion 1011 and the second portion 1012 in FIG. 5A can refer to the description of the first portion 1011 and the second portion 1012 in FIGS. 3A-3C. Since the first portion 1011 and the second portion 1012 are also axisymmetric about the symmetry axis QQ′, the microcavity 401 formed by the first portion 1011, the second portion 1012 and the third portion 1013 is axisymmetric about the symmetry axis QQ′.

As illustrated in FIG. 5B, as an example, when the shapes of the first portion 1011 and the second portion 1012 are circular truncated cones, the shape of the third portion 1013 is a curved surface body. The third portion 1013 comprises a fifth top opening 114 and a sixth bottom opening 115, and the fifth top opening 114 and the sixth bottom opening 115 are both circular. The term “curved surface body” means that any curved surface geometry can be called a curved surface body or a curved surface solid as long as a curved surface is involved. The surface of the curved surface body can be composed entirely of curved surfaces, such as cylinders, spheres, and the like. The surface of the curved surface body can also be a combination of a curved surface and a flat surface. As illustrated, the second bottom opening 111 of the first portion 1011 of the microcavity 401 is the fifth top opening 114 of the third portion 1013, the third top opening 112 of the second portion 1012 of the microcavity 401 is the sixth bottom opening 115 of the third portion 1013.

As illustrated in FIG. 5B, the sidewall of the third portion 1013 is an arc surface with a certain curvature, and the arc surface is more outwardly protruding relative to the fifth top opening 114 of the third portion 1013. The vertical distance S from any point on the sidewall of the third portion 1013 to the reference line BB′ is greater than the radius R of the fifth top opening 114 of the third portion 1013. The reference line BB′ penetrates through the center O of the fifth top opening 114 and the center O′ of the sixth bottom opening 115 of the third portion 1013 and is perpendicular to the reference plane. In an example, the ratio of the maximum value of the vertical distance S (e.g., the vertical distance from the intersection of the sidewall of the third portion 1013 with the symmetry axis QQ′ to the reference line BB′) and the radius R of the fifth top opening 114 is 1.2:1. In a conventional microcavity with a vertical sidewall (e.g. a cylindrical microcavity), the vertical distance S from any point on the sidewall of the microcavity to the reference line BB′ is always equal to the radius R of the opening of the microcavity. In the microcavity 401 provided by the embodiment of the present disclosure, the shape of the microcavity 401 is specially designed so that the ratio of the vertical distance S from a point on the sidewall of the microcavity to the reference line BB′ and the radius R of the opening of the microcavity varies with the position of the point. Such a shape design can make the sample solution flowing on the hydrophobic layer 105 easier to enter the microcavity 401 and keep stably in the microcavity 401.

Similar to the microcavity 301 in FIG. 4A, the sidewall 102 of the microcavity 401 in FIG. 5A has a slope angle with respect to the reference plane. By making the sidewall 102 have an inclination angle with respect to the reference plane, it is favorable for the sample solution to flow into the microcavity 401 along the inclined sidewall 102 of the microcavity 401, so that the sample solution can fully fill each microcavity 401. In addition, the microcavity 401 in FIG. 5A has the middle portion 1013. The existence of the middle portion 1013 can increase the volume of the microcavity 401 on the one hand, so that each microcavity 401 can accommodate much sample solution, more doses of reagents to be tested can be obtained after PCR amplification reaction, so that the minimum detection limit of the microfluidic substrate 400 can be improved. On the other hand, the sidewall of the third portion 1013 is an arc-shaped surface that protrudes outward, so that the sample solution entering the inside of the microcavity 401 is more difficult to flow out of the microcavity 401 along the sidewall. Therefore, the structural design of the microcavity 401 can not only make it easier for the sample solution to enter the inside of the microcavity 401 and fully fill each microcavity 401, but also make the sample solution entering the microcavity 401 keep stably in the microcavity 401 during the detection process, and be not easily taken out of the microcavity 401.

The thickness of the substrate 10 is about 300 μm, that is, the depth of the microcavity 401 is about 300 μm. In some embodiments, the density of the microcavities 401 of the microfluidic substrate 400 is about 9000/cm². In the embodiment in which the top opening 103 of the microcavity 401 is circular, the diameter of the top opening 103 is 110-130 μm, such as 110 μm, 120 μm, 130 μm. The size of the microfluidic substrate 400 can be any appropriate size, and the number of the microcavities 401 can be any appropriate number. The embodiments of the present disclosure do not specifically limit the size of the microfluidic substrate 400 and the number of the microcavities 401. In an example, the size of the microfluidic substrate 400 is 5 cm*5 cm, and the number of the microcavities 401 is 100*100.

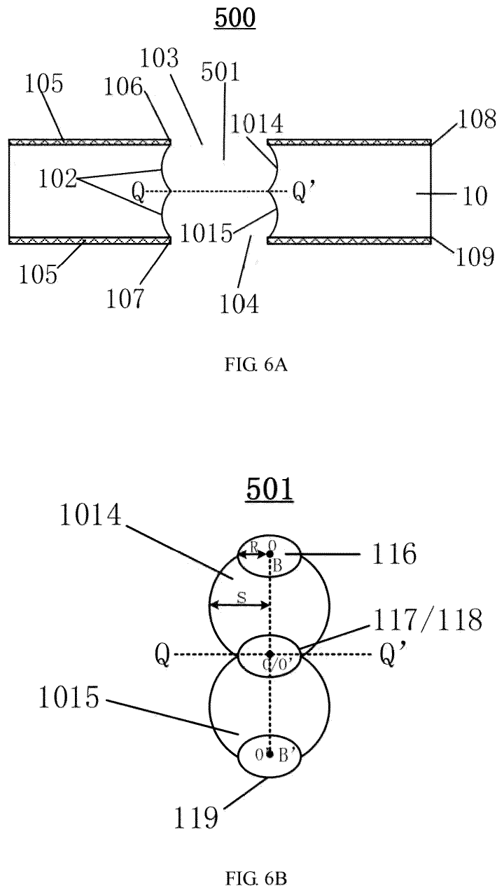

FIG. 6A illustrates a cross-sectional view of a portion of a microfluidic substrate 500 comprising a plurality of microcavities 501. FIG. 6B illustrates a shape of the microcavity 501 as an example. The microfluidic substrate 500 illustrated in FIGS. 6A-6B has substantially the same construction as the microfluidic substrate 200 illustrated in FIGS. 3A-3C, and therefore the same reference numerals are used to refer to the same components. Therefore, the detailed functions of the components in FIGS. 6A-6B with the same reference numerals as those in FIGS. 3A-3C can refer to the description of FIGS. 3A-3C, which will not be repeated here, and only the differences will be described below.

As illustrated in FIGS. 6A-6B, each microcavity 501 comprises a fourth portion 1014 and a fifth portion 1015 stacked on each other and penetrating through each other, the fourth portion 1014 and the fifth portion 1015 are axisymmetric about the symmetry axis QQ′, so that the microcavity 501 is axisymmetric. The shape of the fourth portion 1014 and the fifth portion 1015 is a curved surface body, and the shape of the curved surface body is substantially the same as the shape of the curved surface body of the third portion 1013 in FIG. 5B. The fourth portion 1014 comprises a seventh top opening 116 and an eighth bottom opening 117, the fifth portion 1015 comprises a ninth top opening 118 and a tenth bottom opening 119, and the eighth bottom opening 117 and the ninth top opening 118 are the same opening. The seventh top opening 116 of the fourth portion 1014 is the top opening 103 of the microcavity 501, and the tenth bottom opening 119 of the fifth portion 1015 is the bottom opening 104 of the microcavity 501. The seventh top opening 116 and the eighth bottom opening 117 of the fourth portion 1014 and the ninth top opening 118 and the tenth bottom opening 119 of the fifth portion 1015 are circular.

Similar to FIG. 5B, the sidewalls of the fourth portion 1014 and the fifth portion 1015 in FIG. 6B are arc surfaces with a certain curvature, and the arc surfaces are outwardly convex relative to the seventh top opening 116 of the fourth portion 1014. The vertical distance S from any point on the sidewall of the fourth portion 1014 or the fifth portion 1015 to the reference line BB′ is greater than the radius R of the seventh top opening 116 of the fourth portion 1014. The reference line BB′ penetrates through the center O of the seventh top opening 116 of the fourth portion 1014 and the center O′ of the tenth bottom opening 119 of the fifth portion 1015 and is perpendicular to the reference plane. In an example, the ratio of the maximum value of the vertical distance S to the radius R of the seventh top opening 116 is 1.2:1. In a conventional microcavity with a vertical sidewall (e.g. a cylindrical microcavity), the vertical distance S from any point on the sidewall of the microcavity to the reference line BB′ is always equal to the radius R of the opening of the microcavity. In the microcavity 501 provided by the embodiment of the present disclosure, the shape of the microcavity 501 is specially designed so that the ratio of the vertical distance S from a point on the sidewall of the microcavity to the reference line BB′ to the radius R of the opening of the microcavity varies with the position of the point. Such a structural design can make it easier for the sample solution flowing on the hydrophobic layer 105 to enter the microcavity 501 and keep stably in the microcavity 501.

The fourth portion 1014 and the fifth portion 1015 of the microcavity 501 are both curved surface bodies, so that the sidewall of the microcavity 501 is a curved surface that protrudes outward. Such shape design makes it more difficult for the sample solution entering the inside of the microcavity 501 to flow out to the outside of the microcavity 501 along the sidewall. Therefore, the structural design of the microcavity 501 can make the sample solution entering the microcavity 501 keep stably in the microcavity 501 during the detection process and not easily be taken out of the microcavity 501.

The thickness of the substrate 10 is about 300 μm, that is, the depth of the microcavity 501 is about 300 μm. In some embodiments, the density of the microcavities 501 of the microfluidic substrate 500 is about 3500/cm². The diameter of the top opening 103 of the microcavity 501 is 210-230 μm, such as 210 μm, 220 μm, 230 μm.

The above embodiments describe that the microfluidic substrate comprises a plurality of microcavities, and each microcavity of the plurality of microcavities is a through hole. In an alternative embodiment, a portion of the plurality of microcavities of the microfluidic substrate may be through holes, while the remaining portions may be blind holes.

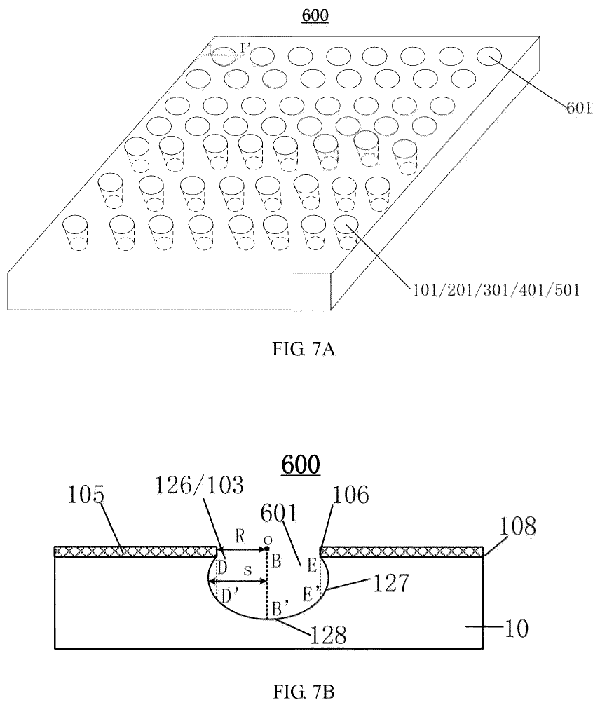

FIG. 7A illustrates a plan view of a microfluidic substrate 600, the microfluidic substrate 600 comprises a plurality of microcavities, a portion of the plurality of microcavities are through holes, and the microcavities in the shape of through hole may be the microcavities 101, 201, 301, 401, 501 described in the previous embodiments; another portion of the plurality of microcavities are blind holes 601. Although FIG. 7A illustrates that the microcavities in the shape of through hole are arranged adjacently and the microcavities in the shape of blind hole are arranged adjacently, this is only an example. The arrangement of through hole microcavity and blind hole microcavity can be flexibly selected according to actual needs. For example, in an alternative embodiment, the through hole microcavities and the blind hole microcavities may be alternately arranged.

FIG. 7B illustrates a cross-sectional view taken along line II′ in FIG. 7A, in which only one blind hole microcavity 601 is illustrated. The shape of the blind hole microcavity 601 is a curved surface body, and the shape of the curved surface body can be referred to the description of the curved surface body in FIG. 5B and FIG. 6B. Different from the curved surface body in FIGS. 5B and 6B, since the microcavities 401 and 501 are through holes in FIGS. 5B and 6B, the curved surface body comprises the top opening, the bottom opening, and the sidewall connecting the top opening and the bottom opening. In FIG. 7B, since the microcavity 601 is a blind hole, the curved surface body comprises an opening 126 and a sidewall 127, and also comprises a bottom 128. The sidewall 127 and the bottom 128 together constitute the reaction cavity of the microcavity 601 to accommodate the sample solution. The opening 126 of the curved surface body is the top opening 103 of the microcavity 601 and is in the shape of a circle, and the circle has a center O. In FIG. 7B, the portion of the microcavity 601 that intersects the dashed line segments DD′ and EE′ is the sidewall 127 of the microcavity 601, and the remaining bottom portion is the bottom 128 of the microcavity 601.

As illustrated in FIG. 7B, the sidewall 127 of the microcavity 601 is to an arc-shaped surface with a certain curvature, and the arc-shaped surface protrudes outwardly relative to the top opening 103 of the microcavity 601. The vertical distance S from any point on the sidewall 127 of the microcavity 601 to the reference line BB′ is greater than the radius R of the top opening 103 of the microcavity 601. The reference line BB′ penetrates through the center O of the top opening 103 of the microcavity 601 and is perpendicular to the reference plane. In an example, the ratio of the maximum value of the vertical distance S (e.g., the vertical distance from the maximum arc of the sidewall 127 to the reference line BB′) to the radius R of the top opening 103 is 1.2:1. In a conventional microcavity with a vertical sidewall (e.g. a cylindrical microcavity), the vertical distance S from any point on the sidewall of the microcavity to the reference line BB′ is always equal to the radius R of the opening of the microcavity. In the microcavity 601 provided by the embodiment of the present disclosure, the shape of the microcavity 601 is specially designed so that the ratio of the vertical distance S from a point on the sidewall of the microcavity to the reference line BB′ to the radius R of the opening of the microcavity varies with the position of the point. Such a structural design can make it easier for the sample solution to enter the microcavity 601 and keep stably in the microcavity 601.

Since the microcavity 601 is a blind hole and the sidewall has a certain curvature, after the sample solution flows into the microcavity 601, it can be kept stably in the microcavity 601 and not easily taken out of the microcavity 601 during the detection process. In addition, if bubbles are generated during the flow of the sample solution into the microcavity 601, the microcavity 601 can adsorb these bubbles on the sidewall 127 to avoid mixing the bubbles with the sample solution in the microcavity 601, thereby avoiding affecting the subsequent fluorescence detection of the sample solution.

In some embodiments, the depth of the microcavity 601 is 50-100 μm, e.g., 50 μm, 75 μm, 100 μm. In some embodiments, the diameter of the top opening 103 of the microcavity 601 is 110-130 μm, such as 110 μm, 120 μm, 130 μm.

As illustrated in FIG. 7B, the microfluidic substrate 600 further comprises a hydrophobic layer 105, and the hydrophobic layer 106 is only disposed on the first surface 108 of the microfluidic substrate 600. The shape of the first via 106 of the hydrophobic layer 105 is adapted to the shape of the top opening 103 of the microcavity 601, and the orthographic projection of the top opening 103 of each microcavity 601 on the reference plane overlaps (e.g. completely overlaps) with the orthographic projection of the first via 106 corresponding to the microcavity 601 on the reference plane.

In the embodiment illustrated in FIGS. 7A and 7B, the microfluidic substrate 600 comprises a plurality of microcavities 601, some of the plurality of microcavities 601 are through holes, and the through hole microcavity can be the microcavity 101, 201, 301, 401, 501 described in any of the previous embodiments. Another of the plurality of microcavities 601 are blind holes, that is, the microcavities 601. Therefore, the microfluidic substrate 600 combines all the advantages of the through hole microcavity and the blind hole microcavity, which not only facilitates the rapid entry of the sample solution into the microcavity, but also enables the sample solution to be kept stably in the microcavity without not easily taken out of the microcavity.

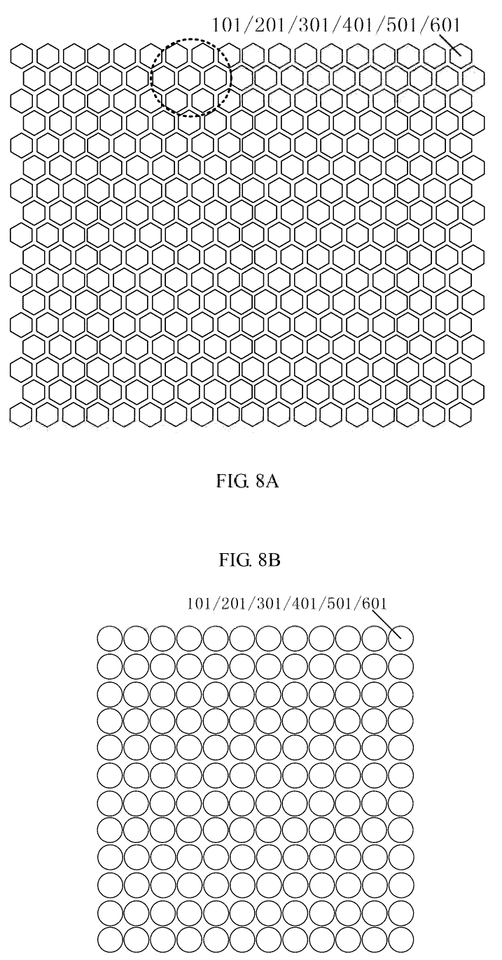

FIG. 8A illustrates an arrangement of a plurality of microcavities on a microfluidic substrate, the plurality of microcavities may be the microcavities 101, 201, 301, 401, 501, 601 described in any of the previous embodiments or any combination thereof. In this figure, the opening of the microcavity is exemplified by a regular hexagon. As illustrated in the figure, the plurality of microcavities are arranged on the microfluidic substrate in a two-dimensional hexagonal close-packed manner, and the distance between any two adjacent microcavities in the plurality of microcavities is 20-50 μm, such as 20 μm, 30 μm, 40 μm, 50 μm. The term “two-dimensional hexagonal close-packed” means that the plurality of microcavities are arranged in a honeycomb-like arrangement on the microfluidic substrate to maximize the use of space area. However, it is necessary to ensure that an appropriate interval is between two adjacent microcavities to avoid mutual interference between the microcavities. As illustrated in the dotted box in FIG. 8A, the two-dimensional hexagonal close-packed arrangement makes the line connecting the centers of the adjacent six microcavities form a regular hexagon, and another microcavity is arranged in the center of the regular hexagon, and the center of the microcavity at the center is coincident with the center of the regular hexagon.

FIG. 8B illustrates another arrangement of a plurality of microcavities on the microfluidic substrate, and the plurality of microcavities may be the microcavities 101, 201, 301, 401, 501, and 601 described in any of the previous embodiments or any combination thereof. In this figure, the opening of the microcavity is exemplified by a circle. As illustrated in the figure, the plurality of microcavities are arranged on the microfluidic substrate in a two-dimensional square lattice, and the distance between any two adjacent microcavities in the plurality of microcavities is 20-50 μm, for example, 20 μm, 30 μm, 40 μm, 50 μm. The term “two-dimensional square lattice” refers to that the plurality of microcavities are regularly arranged on the microfluidic substrate, and the intersection of two adjacent rows of microcavities and two adjacent columns of microcavities is four microcavities, and the line connecting the centers of the bottoms of the four microcavities forms a square. This arrangement of the microcavities can maximize the utilization of the space area, but at the same time ensure that an appropriate interval is between two adjacent microcavities to avoid mutual interference between the microcavities.

During the dPCR reaction, the double-stranded structure of the DNA fragment is denatured at high temperature (e.g., 90° C.) to form a single-stranded structure, the primer and the single strand are combined at low temperature (for example, 65° C.), according to the principle of base complementary pairing. The base-binding extension is achieved at the optimum temperature (e.g. 72° C.) for DNA polymerase. The above process is the temperature cycle process of denaturation-annealing-extension. By multiple temperature cycles of denaturation-annealing-extension, DNA fragments can be replicated in large-scale. In order to realize the above temperature cycle process, in the related art, a series of external devices need to be used to heat and cool the microfluidic device, which makes the device bulky, complicated in operation, low in integration of the microfluidic device and expensive.

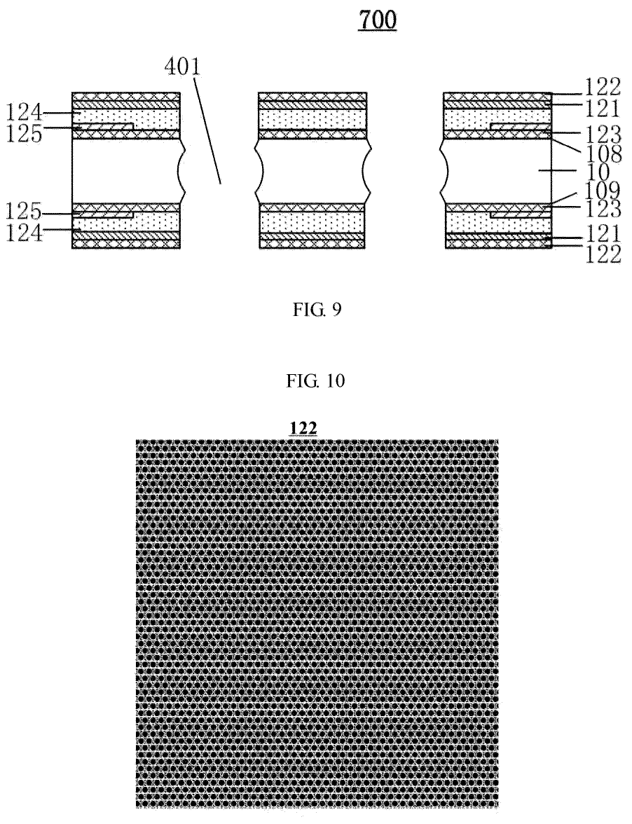

In order to solve the above problems, as illustrated in FIG. 9, an embodiment of the present disclosure provides a microfluidic substrate 700, the microfluidic substrate 700 comprises microcavities and a heating electrode 121, and the microcavities may be any one of the microcavities 101, 201, 301, 401, 501, 601 described in the previous embodiments, or any combination thereof, and have corresponding advantages. FIG. 9 is introduced by taking the microfluidic substrate 700 comprising the microcavity 401 as an example.

The heating electrode 121 may be located on at least one of the opposite first surface 108 and the second surface 109 of the substrate 10 and located in the region between adjacent two microcavities 401. For example, the heating electrode 121 may be located only on the first surface 108 of the substrate 10, or only on the second surface 109 of the substrate 10, or on both opposite first surface 108 and second surface 109 of the substrate 10. The heating electrode 121 is configured to heat the microcavity 401 to provide a suitable temperature for the reaction of the sample solution within the microcavity 401. FIG. 9 illustrates that the heating electrode 121 is located on the opposite first surface 108 and the second surface 109 of the substrate 10 and is located in the region between two adjacent microcavities 401. The heating electrode 121 arranged on both sides of the substrate 10 can provide a better heating effect. The heating electrode 121 can receive an electrical signal (e.g., a voltage signal), whereby when an electric current flows through the heating electrode 121, heat is generated, which heat can be conducted to the adjacent microcavities 401 for polymerase chain reaction. For example, the heating electrode 121 can be made of a conductive material with a relatively high resistivity, so that the heating electrode 121 can generate a relatively large amount of heat when a relatively small electrical signal is supplied to improve the energy conversion rate. The heating electrode 121 can be made of, for example, a transparent conductive material, such as indium tin oxide (ITO), tin oxide, etc., or other suitable materials, such as metal, etc., which are not limited in the embodiments of the present disclosure.

In the embodiment of the present disclosure, by integrating the heating electrode 121 in the microfluidic substrate 700, the heating of the microcavity 401 of the microfluidic substrate 700 can be effectively realized, thereby realizing the temperature control of the microcavity 401 without external heating device, so the integration is high, the volume is small, the operation is simple, and the cost can be reduced.

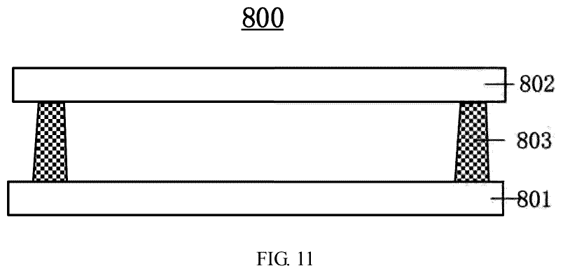

As illustrated in FIG. 9, the microfluidic substrate 700 may further comprise a hydrophobic layer 122, wherein the hydrophobic layer 122 is located on a side of the heating electrode 121 away from the first surface 108 and on a side of the heating electrode 121 away from the second surface 109. The hydrophobic layer 122 has hydrophobic and lipophilic property, which is beneficial for the aqueous sample solution flowing thereon to flow into the microcavity 401. FIG. 10 illustrates a schematic structural diagram of the hydrophobic layer 122, wherein the circular opening is the microcavity 401, and the hydrophobic layer 122 is arranged in the region between two adjacent microcavities 401. The material of the hydrophobic layer 122 may be resin or silicon nitride. In an example, the material of the hydrophobic layer 122 is SiN.

The microfluidic substrate 700 may further comprise a conductive layer 125, the conductive layer 125 is located on a side of the heating electrode 121 close to the first surface 108 and a side of the heating electrode 121 close to the second surface 109, and is arranged around the periphery of the microfluidic substrate 700. The conductive layer 125 is electrically connected to the heating electrode 121. The conductive layer 125 is configured to apply an electrical signal (e.g., a voltage signal) to the heating electrode 121, and after receiving the electrical signal, the heating electrode 121 can generate heat under the action of the electrical signal, thereby heating the microcavity 401. The resistance value of the heating electrode 121 may be greater than that of the conductive layer 125, so that under the action of the same electrical signal, the heating electrode 121 generates more heat and the conductive layer 125 generates less heat, thereby reducing energy loss. For example, the conductive layer 125 can be made of a material with lower resistivity, thereby reducing the energy loss on the conductive layer 125. The conductive layer 125 may be made of a metal material, for example, the metal material may be molybdenum (Mo), copper or copper alloy, aluminum or aluminum alloy, etc., and may be a single metal layer or a composite metal layer, which is not limited in the embodiments of the present disclosure.

The microfluidic substrate 700 may further comprise a first dielectric layer 123 and a second dielectric layer 124. The first dielectric layer 123 is located on the side of the heating electrode 121 close to the first surface 108 and the side of the heating electrode 121 close to the second surface 109, the second dielectric layer 124 is located on a side of the first dielectric layer 123 away from the first surface 108 and a side of the first dielectric layer 123 away from the second surface 109. The conductive layer 125 is located between the first dielectric layer 123 and the second dielectric layer 124, and may be electrically connected to the heating electrode 121 via through holes in the second dielectric layer 124. The first dielectric layer 123 and the second dielectric layer 124 may be any suitable material, which is not limited by the embodiment of the present disclosure. In an example, the material of the first dielectric layer 123 is SiN, and the material of the second dielectric layer 124 is SiO.

According to another aspect of the present disclosure, a microfluidic chip is provided. FIG. 11 illustrates a schematic structural diagram of a microfluidic chip 800. The microfluidic chip 800 comprises a microfluidic substrate 801, and the microfluidic substrate 801 may be any of the microfluidic substrates 100, 200, 300, 400, 500, 600, 700 described in the previous embodiments.

The microfluidic chip 800 may further comprise a counter substrate 802 assembled with the microfluidic substrate 801 and an encapsulant 803 located between the microfluidic substrate 801 and the counter substrate 802.

In an example, both the microfluidic substrate 801 and the counter substrate 802 comprise glass substrates. The microfluidic substrate 801 and the counter substrate 802 are arranged opposite to each other, and play the roles of protection, support, isolation and the like. The microfluidic chip 800 is prepared by a glass-based micromachining method combined with a semiconductor process, so that mass production can be realized and the production cost can be greatly reduced.

The encapsulant 803 is configured to seal the microfluidic substrate 801 and the counter substrate 802, and is configured to maintain an appropriate space between the microfluidic substrate 801 and the counter substrate 802 to provide sufficient space for the flow of the sample solution.

The microfluidic chip 800 may have substantially the same technical effects as the microfluidic substrates described in the previous embodiments. Therefore, for the sake of brevity, the description of the technical effects of the microfluidic chip 800 will not be repeated here.

Yet another aspect of the present disclosure provides a method 900 for manufacturing a microfluidic substrate. Different microfluidic substrates described in the above embodiments have basically the same manufacturing steps, there are only some differences in the details of some steps. The following briefly describes the method by taking the microfluidic substrate 100 illustrated in FIGS. 2A-2C as an example.

Step 901: providing the substrate 10 and cleaning it. The substrate 10 can be made of any suitable material, and in an example, the substrate 10 is made of glass. The substrate 10 may have any suitable thickness, and in an example, the thickness of the substrate 10 is 300 μm.

Step 902: preparing a mark on the substrate 10 to provide a positioning function for the subsequent etching of the microcavity and cutting of the substrate. In an example, the process of forming the mark is as follows: under the conditions that the sputtering cavity temperature is about 230° C., the volume flow rate of Ar is about 100 sccm (standard cubic centimeter per minute), the pressure is about 0.3 Pa, the power is about 12 kW, and the scanning frequency is about 15 scan, a metal Mo film with a thickness of about 2,200 Å is sputtered on the surface of the substrate 10, and the Mo film is exposed, developed and etched by photolithography to form a metal mark.

Step 903: depositing an insulating film on the first surface 108 of the substrate 10, and performing exposure, develop and etching on the insulating film to form the hydrophobic layer 105. In an example, the process of forming the hydrophobic layer 105 is as follows: in a plasma enhanced chemical vapor deposition (PECVD) apparatus, under the conditions that the temperature is about 390° C., the power is about 600 W, the pressure is about 1200 mtorr, and the distance between the plasma reaction enhancing target and the sample to be deposited in the PECVD apparatus is about 1000 mils, SiH₄ (the volume flow rate is about 140 sccm), NH₃ (the volume flow rate is about 700 sccm) and N₂ (the volume flow rate is about 2260 sccm, and the introduction time is about 225 seconds) are introduced into the reaction cavity to deposit a SiNₓ film with a thickness of about 3000 Å on the first surface 108 of the substrate 10. The SiNₓ film is exposed, developed and etched to form the hydrophobic layer 105.

Step 904: forming a first metal mask on a side of the hydrophobic layer 105 away from the first surface 108. The first metal mask is used to provide isolation and protection for other portions of the microfluidic substrate except the microcavities during the subsequent etching of the microcavities. In an example, the process of forming the first metal mask is as follows: under the conditions that the sputtering cavity temperature is about 230° C., the volume flow rate of Ar is about 100 sccm, the pressure is to about 0.3 Pa, the power is about 12 kW, and the scanning frequency is about 15 scan, a metal Mo film with a thickness of about 2,200 Å is sputtered on the side of the hydrophobic layer 105 away from the first surface 108, and the Mo film is exposed, developed and etched by photolithography to form the first metal mask. The first metal mask comprises a plurality of vias, and the plurality of vias correspond to the positions of a plurality of microcavities to be formed later and have the same shape, so as to expose the regions needing to be etched later to form microcavities.

Step 905: sequentially forming a hydrophobic layer 105 and a second metal mask on the second surface 109 of the substrate 10 by marking and aligning. The position of the hydrophobic layer 105 on the second surface 109 completely corresponds to the position of the hydrophobic layer 105 on the first surface 108, and the position of the second metal mask on the second surface 109 completely corresponds to the position of the first metal mask on the first surface 108. The preparation method of the hydrophobic layer 105 and the second metal mask on the second surface 109 is exactly the same as steps 903 and 904.

Step 906: etching the substrate 10 by dry etching to form a plurality of microcavities 101 which are all through holes. In an example, the process of forming a plurality of microcavities 101 by dry etching is as follows: using inductively coupled plasma etching (ICP) method, under the conditions that the power in the reaction cavity is about 2500 W, the temperature is about 20° C., the pressure is about 0.6 Pa, the flow rate of C₄F₈ is about 60 ml/min, the flow rate of Ar is about 120 ml/min, and the etching rate is about 0.8 μm/min, the substrate 10 is etched for about 375 minutes to form a plurality of microcavities 101. The shape of the top opening 103 of the microcavity 101 formed by dry etching may be circular or regular polygon. When the top opening 103 of the microcavity 101 is circular, the diameter of the top opening 103 is about 110-130 μm, for example, 120 μm, the density of the microcavities 101 is about 9,000/cm², the depth of microcavity 101 is 300 μm, and the distance between two adjacent microcavity 101 is 20-50 μm. The sidewall 102 of the microcavity 101 has a certain inclination angle, which is favorable for the sample solution to fully fill the microcavity 101 when the sample solution flows. The specific technical effect of the microcavity 101 may refer to the previous description of FIGS. 2A-2C, which will not be repeated here.

Step 907: removing the first metal mask and the second metal mask after etching the microcavity 101.

Step 908: placing the microfluidic substrate 100 on the base, and sliding the sample solution in the same direction with a special instrument (such as a doctor blade) to fill the microcavity 101. After filling, the mineral oil is dripped into the top opening 103 of the microcavity 101. At this time, the encapsulation glue is fixed on the base, and the counter substrate is fixed on the encapsulation glue to form the microfluidic device. Then mineral oil is filled from the injection hole of the microfluidic device, and the injection hole is closed, so as to realize the packaging of the microfluidic device.

The manufacturing method of the microfluidic substrate 200 illustrated in FIGS. 3A-3C is basically the same as the manufacturing method of the microfluidic substrate 100 illustrated in FIGS. 2A-2C, except for the differences in some steps. The same method steps may refer to the description of the manufacturing method of the microfluidic substrate 100, and only the differences of the manufacturing method of the microfluidic substrate 200 will be described below.

The microfluidic substrate 200 is prepared using exactly the same method steps and fabrication sequence as steps 901-905.

Then in step 906A, the etching method of the microcavity 201 of the microfluidic substrate 200 is slightly different from the etching method of the microcavity 101 of the microfluidic substrate 100. The substrate 10 is also etched by dry etching, and the process of forming the plurality of microcavities 201 is as follows: using the ICP method, under the conditions that the power in the reaction cavity is about 2500 W, the temperature is about 20° C., the pressure is about 0.6 Pa, the flow rate of C₄F₈ is about 60 ml/min, the flow rate of Ar is about 120 ml/min, and the etching rate is about 0.8 μm/min, one side of the substrate 10 is first etched for about 188 minutes to form the first portion 1011 of the microcavity 201 in the substrate 10; then the other side of the substrate 10 is etched for about 188 minutes to form the second portion 1012 of the microcavity 201 in the substrate 10, thereby forming the plurality of microcavities 201. The shape of the top opening 103 of the microcavity 201 formed by dry etching may be a circle or a regular polygon. When the top opening 103 of the microcavity 201 is circular, the diameter of the top opening 103 is about 110-130 μm, for example, 120 μm, the density of the microcavities 201 is about 9000/cm², the depth of the microcavity 201 is 300 μm, and the distance between the adjacent two microcavities 201 is 20-50 μm. The sidewall 102 of the microcavity 201 has a certain inclination angle, which is favorable for the sample solution to fully fill the microcavity 201 during the injection. For the specific technical effect of the microcavity 201, reference may be made to the foregoing descriptions of FIGS. 3A-3C, which will not be repeated here.

The microfluidic substrate 200 is then prepared using exactly the same method steps and fabrication sequence as steps 907-908 to complete the encapsulation.

The manufacturing method of the microfluidic substrate 300 illustrated in FIGS. 4A-4C is basically the same as the manufacturing method of the microfluidic substrate 100 illustrated in FIGS. 2A-2C, with only differences in some steps. For the same method steps, reference may be made to the description of the manufacturing steps of the microfluidic substrate 100, and only the differences in the manufacturing method of the microfluidic substrate 300 will be described below.

The microfluidic substrate 300 is prepared using exactly the same method steps and fabrication sequence as steps 901-905.

Then in step 906B, the etching method of the microcavity 301 of the microfluidic substrate 300 is slightly different from the etching method of the microcavity 101 of the microfluidic substrate 100. The substrate 10 is to also etched by dry etching, and the process of forming a plurality of microcavities 301 is as follows: using the ICP method, under the conditions that the power in the reaction cavity is about 2500 W, the temperature is about 20° C., the pressure is about 0.6 Pa, the flow rate of C₄F₈ is about 60 ml/min, the flow rate of Ar is about 120 ml/min, and the etching rate is about 0.8 μm/min, one side of the substrate 10 is first etched for about 125 minutes to form the first portion 1011 of the microcavity 301 in the substrate 10, then the other side of the substrate 10 is etched for about 125 minutes to form the second portion 1012 of the microcavity 301 in the substrate 10. Then, by using laser etching, a suitable laser spot is selected and positioned at the center of each microcavity for ablation to form the third portion 1013, thereby forming the plurality of microcavities 301. The shape of the top opening 103 of the microcavity 301 formed by dry etching may be a circle or a regular polygon. When the top opening 103 of the microcavity 301 is circular, the diameter of the top opening 103 is about 110-130 μm, for example, 120 μm, the density of the microcavities 301 is about 9000/cm², the depth of the microcavity 301 is 300 μm, and the distance between the adjacent two microcavities 301 is 20-50 μm. The sidewall 102 of the microcavity 301 has a certain inclination angle, which is beneficial for the sample solution to fully fill the microcavity 301 during the injection. For the specific technical effect of the microcavity 301, reference may be made to the foregoing descriptions about FIGS. 4A-4C, which will not be repeated here.

The microfluidic substrate 300 is then prepared using the exact same method steps and fabrication sequence as steps 907-908 to complete the encapsulation.

The manufacturing method of the microfluidic substrate 400 illustrated in FIGS. 5A-5B is basically the same as the manufacturing method of the microfluidic substrate 100 illustrated in FIGS. 2A-2C, with only differences in some steps. For the same method steps, reference may be made to the description of the manufacturing steps of the microfluidic substrate 100, and only the differences in the manufacturing method of the microfluidic substrate 400 will be described below.

The microfluidic substrate 400 is prepared using exactly the same method steps and fabrication sequence as steps 901-905.

Then in step 906C, the etching method of the microcavity 401 of the microfluidic substrate 400 is slightly different from the etching method of the microcavity 101 of the microfluidic substrate 100. The process of forming a plurality of microcavities 401 is as follows: first, using the ICP method, under the conditions that the power in the reaction cavity is about 2500 W, the temperature is about 20° C., the pressure is about 0.6 Pa, the flow rate of C₄F₈ is about 60 mi/min, the flow rate of Ar is about 120 ml/min, and the etching rate is about 0.8 um/min, one side of the substrate 10 is first etched for about 60 minutes to form the first portion 1011 of the microcavity 401 in the substrate 10, and then the other side of the substrate 10 is etched for about 60 minutes to form the second portion 1012 of the microcavity 401 in the substrate 10. Then, a third metal mask is formed on the microfluidic substrate 400. The third metal mask is used to provide isolation and protection for the already formed first and second portions and other portions other than the microcavities of the microfluidic substrate during the subsequent etching of the third portion of the microcavity. In an example, the process of forming the third metal mask is as follows: under the conditions that the temperature of the sputtering cavity is about 230° C., the volume flow of Ar is about 100 sccm, the pressure is about 0.3 Pa, the power is about 12 kW, the scanning frequency is about 15 scans, a metal Mo film with a thickness of about 2200 Å is sputtered on the microfluidic substrate 400, and the Mo film is exposed, developed and etched by a photolithography process to form the third metal mask. The third metal mask covers the region that needs to be protected, and exposes the third portion of the microcavity that needs to be etched later. Then, the third portion 1013 of the microcavity 401 is formed using wet etching. The specific steps can be described as: the microfluidic substrate 400 is soaked in an etching solution, the concentration of hydrogen fluoride (HF) in the etching solution is about 40%, the etching to rate is about 3.5 um/min. During the etching, the etching solution is continuously stirred by the blade, so that the etching solution can etch the substrate 10 of the microfluidic substrate 400 more uniformly. The etching time is about 30 minutes to etch the third portion 1013 of the microcavity 401, thereby forming the microcavity 401. The microcavity 401 is formed by a combination of dry etching and wet etching. Due to the isotropic properties of wet etching, the shape of the top opening 103 of the microcavity 401 is generally circular, and the diameter of the top opening 103 is about 110-130 μm, such as 120 μm, the density of the microcavities 401 is about 9000/cm², the depth of the microcavity 401 is 300 μm, and the distance between two adjacent microcavities 401 is 20-50 μm. The sidewall 102 of the microcavity 401 has a certain inclination angle, which is beneficial for the sample solution to fully fill the microcavity 401 during injection, and the sample solution is easy to be kept stably during the detection process and is not easily taken out of the microcavity 401. For the specific technical effect of the microcavity 401, reference may be made to the foregoing description of FIGS. 5A-51B, which will not be repeated here.

Then in step 907C, after the etching of the microcavity 401 is completed, the first metal mask, the second metal mask and the third metal mask are removed.

Finally, the same method step as in step 908 is used to prepare the microfluidic substrate 400 to complete the encapsulation.

The manufacturing method of the microfluidic substrate 500 illustrated in FIGS. 6A-6B is basically the same as the manufacturing method of the microfluidic substrate 100 illustrated in FIGS. 2A-2C, with only differences in some steps. For the same method steps, reference may be made to the description of the manufacturing steps of the microfluidic substrate 100, and only the differences in the manufacturing method of the microfluidic substrate 500 will be described below.

The microfluidic substrate 500 is prepared using exactly the same method steps and fabrication sequence as steps 901-905.

Then in step 906D, the etching method of the microcavity 501 of the microfluidic substrate 500 is slightly different from the etching method of the microcavity 101 of the microfluidic substrate 100. The process of forming the plurality of microcavities 501 is as follows: the microcavities 501 are formed by using a wet etching method. The specific steps can be described as: the microfluidic substrate 500 is soaked in an etching solution, the concentration of hydrogen fluoride (HF) in the etching solution is about 40%, and the etching rate is about 3.5 um/min. The first surface 108 and the second surface 109 are simultaneously etched. During the etching, the etching solution is continuously stirred by the blade, so that the etching solution can etch the substrate 10 of the microfluidic substrate 500 more uniformly. The etching time is about 60 minutes to etch the fourth portion 1014 and the fifth portion 1015 of the microcavity 501, thereby forming the microcavity 501. The microcavity 501 is formed by wet etching. Due to the isotropic properties of wet etching, the shape of the top opening 103 of the microcavity 501 is usually circular, and the diameter of the top opening 103 is about 210-230 μm, for example, 220 μm, the density of the microcavities 501 is about 3500/cm², the depth of the microcavity 501 is 300 μm, and the distance between two adjacent microcavities 501 is 20-50 μm. The structure of the microcavity 501 is beneficial to keep the sample solution stably in the microcavity 501 during the detection process and not easy to be taken out of the microcavity 501. For the specific technical effect of the microcavity 501, reference may be made to the foregoing descriptions about FIGS. 6A-6B, and details are not repeated here.

Finally, the microfluidic substrate 500 is prepared by using exactly the same method steps and manufacturing sequence as steps 907-908 to complete the encapsulation.

The manufacturing method of the microfluidic substrate 600 illustrated in FIGS. 7A-7B is basically the same as the manufacturing method of the microfluidic substrate 100 illustrated in FIGS. 2A-2C, with only differences in some steps. Since the microfluidic substrate 600 comprises a plurality of microcavities, some of the microcavities are through holes, and the other of the microcavities are blind holes. For the fabrication method of the through-hole microcavity, reference may be made to the foregoing description, which will not be repeated here. Only the manufacturing method of the blind-hole microcavity 601 will be described below.

The microfluidic substrate 600 is prepared using exactly the same method steps and fabrication sequence as steps 901-905.

Then in step 906E, the etching method of the microcavity 601 of the microfluidic substrate 600 is slightly different from the etching method of the microcavity 101 of the microfluidic substrate 100. The process of forming the plurality of microcavities 601 is as follows: the microcavities 601 are formed by using a wet etching method. The specific steps can be described as: the microfluidic substrate 600 is soaked in an etching solution, the concentration of hydrogen fluoride (HF) in the etching solution is about 40%, and the etching rate is about 3.5 um/min. The first surface 108 of the substrate 10 is etched. During the etching, the etching solution is continuously stirred by the blades, so that the etching solution can etch the substrate 10 of the microfluidic substrate 600 more uniformly. The etching time is about 30 minutes to form the microcavity 601, which is a blind hole. The microcavity 601 is formed by wet etching. Due to the isotropic property of wet etching, the shape of the top opening 103 of the microcavity 601 is usually circular, and the diameter of the top opening 103 is about 110-130 μm, for example, 120 μm, the density of the microcavities 601 is about 9000/cm², the depth of the microcavity 601 is about 50-100 μm, and the distance between two adjacent microcavities 601 is about 20-50 μm. The blind hole structure of the microcavity 601 is beneficial to keep the sample solution stably in the microcavity 601 during the detection process and not easy to be taken out of the microcavity 601. For the specific technical effects of the microcavity 601, reference may be made to the foregoing descriptions of FIGS. 7A-7B, which will not be repeated here.

Finally, the microfluidic substrate 600 can be prepared using exactly the same method steps and manufacturing sequence as steps 907-908 to complete the encapsulation. In an alternative embodiment, the encapsulation method in step 908 may also be the following process: on the etched microfluidic substrate 600, a frame is formed around the substrate with UV glue or oil-resistant glue film mixed with spacers. Then the counter substrate is bonded to the microfluidic substrate 600 to form a microfluidic device. After the sample solution is added through the injection hole of the counter substrate, mineral oil is then added through the injection hole, and after the inner space is completely filled, the injection hole and the sample outlet of the microfluidic device are closed.

The manufacturing method of the microfluidic substrate 700 illustrated in FIG. 9 is basically the same as the manufacturing method of the microfluidic substrate 400 illustrated in FIGS. 5A-5B, with only differences in some steps. For the same method steps, reference may be made to the description of the manufacturing steps of the microfluidic substrate 400, and only the differences in the manufacturing method of the microfluidic substrate 700 are described below.

First, the microfluidic substrate 700 is prepared using exactly the same method steps and fabrication sequence as steps 901-905. It should be noted that, here, the hydrophobic layer 105 formed by the steps 903 and 905 does not serve as the hydrophobic layer of the microfluidic substrate 700, but serves as the first dielectric layer 123. The preparation method and material of the first dielectric layer 123 are exactly the same as the preparation method and material of the hydrophobic layer 105 in steps 903 and 905.

Then, the same method as steps 906C and 907C of the microfluidic substrate 400 is used to prepare the microcavity 401 of the microfluidic substrate 700.

After the step 907C, a metal layer is deposited on the side of the first dielectric layer 123 away from the first surface 108 and on the side of the first dielectric layer 123 away from the second surface 109, and the metal layer is patterned to form the conductive layer 125. The conductive layer 125 is arranged around the periphery of the microfluidic substrate 700. In an example, the conductive layer 125 is a stacked structure of Mo—AlNd—Mo, and the thickness of Mo film, AlNd film, and Mo film of Mo—AlNd—Mo is 200 Å, 3000 Å and 800 Å, respectively.

Then, an insulating layer is deposited on the side of the first dielectric layer 123 away from the first surface 108 and on the side of the first dielectric layer 123 away from the second surface 109, and the insulating layer is patterned to form a second dielectric layer 124. The second dielectric layer 124 may be any suitable material. In an example, the material of the second dielectric layer 124 is SiOₓ. In an example, the thickness of the second dielectric layer 124 is 3000 Å.

Then, a conductive film is deposited on a side of the second dielectric layer 124 away from the first surface 108 and on a side of the second dielectric layer 124 away from the second surface 109, and the conductive film is patterned to form the heating electrode 121. The heating electrode 121 is located in the region between two adjacent microcavities. The heating electrode 121 may be any suitable material, and in an example, the material of the heating electrode 121 is indium tin oxide (ITO). In an example, the thickness of the heating electrode 121 is 1350 Å.

Then, an insulating film is deposited on a side of the heating electrode 121 away from the first surface 108 and on a side of the heating electrode 121 away from the second surface 109, and the insulating film is exposed, developed and etched to form the hydrophobic layer 122. In an example, the process of forming the hydrophobic layer 122 is as follows: in a plasma enhanced chemical vapor deposition (PECVD) apparatus, under the conditions that the temperature is about 390° C., the power is about 600 W, the pressure is about 1200 mtorr, and the distance between the plasma reaction enhanced target and the sample to be deposited in the PECVD apparatus is about 1000 mils, SiH₄. (the volume flow is about 140 sccm), NH₃ (the volume flow is about 700 sccm) and N₂ (the volume flow is about 2260 sccm, and the introduction time is about 225 seconds) are introduced into the reaction cavity, so as to deposit a SiNₓ film with a thickness of about 3000 Å on the side of the heating electrode 121 away from the first surface 108 and on the side of the heating electrode 121 away from the second surface 109. The SiNₓ film is exposed, developed and etched to form the hydrophobic layer 122.

Finally, the same method step as in step 908 is used to prepare the microfluidic substrate 700 to complete the encapsulation.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed above could be termed a second element, component, region, layer or section without departing from the teachings of the present disclosure.

Spatially relative terms such as “row”, “column”, “below”, “above”, “left”, “right”, etc. may be used herein for ease of description to describe factors such as the relationship of one element or feature to another element or feature(s) illustrated in the figures. It will be understood that these spatially relative terms are intended to encompass different orientations of the device in use or operation in addition to the orientation depicted in the figures. For example, if the device in the figures is turned over, elements described as “below” other elements or features would then be oriented “above” other elements or features. Thus, the exemplary term “below” can encompass both an orientation of above and below. The device may be otherwise oriented (rotated 90 degrees or at other orientations) and the spatially relative descriptors used herein are interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to limit the present disclosure. As used herein, the singular forms “a,” “an,” and “the” are intended to comprise the plural forms as well, unless the context clearly dictates otherwise. It will be further understood that the terms “comprise” and/or “include” when used in this specification designate the presence of stated features, integers, steps, operations, elements and/or parts, but do not exclude the presence or addition of one or more other features, integers, steps, operations, elements, components and/or groups thereof. As used herein, the term “and/or” comprises any and all combinations of one or more of the associated listed items. In the description of this specification, description with reference to the terms “one embodiment,” “another embodiment,” etc. means that a particular feature, structure, material, or characteristic described in connection with the embodiment is included in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification without conflicting each other.

It will be understood that when an element or layer is referred to as being “on”, “connected to”, “coupled to”, or “adjacent to” another element or layer, it may be directly on, directly connected to, directly coupled to, or directly adjacent to another element or layer, or intervening elements or layers may be present. In contrast, when an element is referred to as being “directly on”, “directly connected to”, “directly coupled to”, “directly adjacent to” another element or layer, with no intervening elements or layers present. However, in no case should “on” or “directly on” be interpreted as requiring a layer to completely cover the layer below.

Embodiments of the disclosure are described herein with reference to schematic illustrations (and intermediate structures) of idealized embodiments of the disclosure. As such, variations to the shapes of the illustrations are to be expected, e.g., as a result of manufacturing techniques and/or tolerances. Accordingly, embodiments of the present disclosure should not be construed as limited to the particular shapes of the regions illustrated herein, but are to comprise deviations in shapes due, for example, to manufacturing. Thus, the regions illustrated in the figures are schematic in nature and their shapes are not intended to illustrate the actual shape of a region of a device and are not intended to limit the scope of the present disclosure.

Unless otherwise defined, all terms (comprising technical and scientific terms) used herein have the same meaning as commonly understood by one of ordinary skill in the art to which this disclosure belongs. It will be further understood that terms such as those defined in commonly used dictionaries should be construed to have meanings consistent with their meanings in the relevant art and/or the context of this specification, and will not be idealized or overly interpreted in a formal sense, unless expressly defined as such herein.

The above descriptions are merely specific embodiments of the present disclosure, but the protection scope of the present disclosure is not limited thereto. Any person skilled in the art can easily think of changes or substitutions within the technical scope disclosed by the present disclosure, which should be comprised within the protection scope of the present disclosure. Therefore, the protection scope of the present disclosure should be based on the protection scope of the claims.

1. A microfluidic substrate comprising:
a plurality of microcavities arranged in an array,
wherein at least some of the plurality of microcavities are through holes, and a tangent plane at each of at least some points on a sidewall of each microcavity forms a non-perpendicular angle with a reference plane where the microfluidic substrate is located.

2. The microfluidic substrate of claim 1, wherein the sidewall of each microcavity comprises at least one of a curved surface and an inclined surface, the inclined surface is non-perpendicular to the reference plane.

3. The microfluidic substrate of claim 1, wherein each of the plurality of microcavities is a through hole, and each microcavity comprises a top opening and a bottom opening.

4. The microfluidic substrate of claim 3, wherein a shape of each microcavity is a circular truncated cone or a regular prismoid, and an area of an orthographic projection of the top opening of each microcavity on the reference plane is larger than an area of an orthographic projection of the bottom opening of each microcavity on the reference plane.

5. The microfluidic substrate of claim 4,
wherein an angle between a normal of any point on the sidewall of each microcavity and a reference line is 82°˜85°, the reference line is perpendicular to the reference plane, and
wherein a shape of the top opening of each microcavity is a circle, and a diameter of the circle is 110˜130 μm.

6. The microfluidic substrate of claim 4, further comprising:
a hydrophobic layer,
wherein the hydrophobic layer is on a first surface and a second surface of the microfluidic substrate which are opposite, a portion of the hydrophobic layer on the first surface comprises a plurality of first vias, and a portion of the hydrophobic layer on the second surface comprises a plurality of second vias, and
wherein the plurality of first vias and the plurality of second vias correspond to the plurality of microcavities one by one respectively, the orthographic projection of the top opening of each of the plurality of microcavities on the reference plane is within an orthographic projection of a first via corresponding to the microcavity on the reference plane, and the orthographic projection of the bottom opening of each of the plurality of microcavities on the reference plane overlaps an orthographic projection of the second via corresponding to the microcavity on the reference plane.

7. The microfluidic substrate of claim 3, wherein a shape of each microcavity is axisymmetric about a symmetry axis, the symmetry axis is parallel to the reference plane.

8. The microfluidic substrate of claim 7,
wherein each microcavity comprises a first portion and a second portion stacked on and penetrating through each other, the first portion and the second portion are axisymmetric about the symmetry axis, and a shape of each of the first portion and the second portion is one of a circular truncated cone and a regular prismoid, and
wherein an area of an orthographic projection of a first top opening of the first portion on the reference plane is larger than an area of an orthographic projection of a second bottom opening of the first portion on the reference plane, an area of an orthographic projection of a third top opening of the second portion on the reference plane is smaller than an area of an orthographic projection of a fourth bottom opening of the second portion on the reference plane.

9. The microfluidic substrate of claim 8, wherein each microcavity further comprises a third portion between the first portion and the second portion and connecting the first portion and the second portion, the second bottom opening of the first portion is a fifth top opening of the third portion, the third top opening of the second portion is a sixth bottom opening of the third portion, and the third portion is axisymmetric about the symmetry axis.

10. The microfluidic substrate of claim 9,
wherein the shape of each of the first portion and the second portion is the circular truncated cone, and a shape of the third portion is a cylinder, or
wherein the shape of each of the first portion and the second portion is a regular quadrangular prismoid, and a shape of the third portion is a cuboid.

11. The microfluidic substrate of claim 9, wherein the shape of each of the first portion and the second portion is the circular truncated cone, and a shape of the third portion is a curved surface body, a vertical distance from any point on a sidewall of the third portion to a reference line is greater than a radius of the fifth top opening of the third portion, the reference line passes through centers of the fifth top opening and the sixth bottom opening of the third portion and is perpendicular to the reference plane.

12. (canceled)

13. The microfluidic substrate of claim 7,
wherein each microcavity comprises a fourth portion and a fifth portion stacked on and penetrating through each other, the fourth portion and the fifth portion are axisymmetric about the symmetry axis, and
wherein a shape of each of the fourth portion and the fifth portion is a curved surface body, a shape of each of the top opening and the bottom opening of each microcavity is a circle, a vertical distance from any point on the sidewall of each microcavity to a reference line is greater than a radius of the top opening, and the reference line passes through centers of the top opening and the bottom opening and is perpendicular to the reference plane.

14. (canceled)

15. The microfluidic substrate of claim 3, wherein a diameter of the top opening is 210˜230 μm, and a depth of each microcavity is 300 μm.

16. The microfluidic substrate of claim 1, wherein others of the plurality of microcavities are blind holes.

17. The microfluidic substrate of claim 16, wherein a shape of the blind hole is a curved surface body, the blind hole comprises an opening, a sidewall and a bottom, the opening of the blind hole is a top opening of the microcavity and a shape of the opening is a circle, a vertical distance from any point on the sidewall of the blind hole to a reference line is greater than a radius of the top opening, the reference line passes through the center of the top opening and is perpendicular to the reference plane.

18. (canceled)

19. The microfluidic substrate of claim 17,
wherein a depth of the blind hole is 50˜100 μm, and a diameter of the opening of the blind hole is 110˜130 μm, and
wherein a ratio of the maximum value of the vertical distance to the radius of the top opening is 1.2:1.

20. (canceled)

21. The microfluidic substrate of claim 1,
wherein a distance between two adjacent microcavities in the plurality of microcavities is 20˜50 μm, and
wherein the plurality of microcavities are arranged in a glass substrate of the microfluidic substrate.

22. The microfluidic substrate of claim 1, further comprising:
a heating electrode,
wherein the heating electrode is arranged in a region between two adjacent microcavities on at least one of a first surface and a second surface of the microfluidic substrate which are opposite.

23. (canceled)

24. The microfluidic substrate of claim 22, further comprising:
a hydrophobic layer, wherein the heating electrode is arranged in the region between two adjacent microcavities on both the first surface and the second surface of the microfluidic substrate which are opposite, and the hydrophobic layer is on a side of the heating electrode away from the first surface and a side of the heating electrode away from the second surface;
a first dielectric layer on a side of the heating electrode close to the first surface and a side of the heating electrode close to the second surface;
a second dielectric layer on a side of the first dielectric layer away from the first surface and a side of the first dielectric layer away from the second surface; and
a conductive layer between the first dielectric layer and the second dielectric layer and arranged on a periphery of the microfluidic substrate, the conductive layer being electrically connected to the heating electrode through vias in the second dielectric layer.

25. A microfluidic chip comprising the microfluidic substrate of claim 1, a counter substrate assembled with the microfluidic substrate, and an encapsulant between the microfluidic substrate and the counter substrate.

26. (canceled)
